(19) United States
(12) Patent Application Publication
(10) Pub. No.: US 2011/0055936 A1
(43) Pub. Date: Mar. 3, 2011

(54) TRANSGENIC MAMALS MODIFIELD IN BRI PROTEIN EXPRESSION

(76) Inventors: Luciano D'Adamio, Bronx, NY (US); Luca Giliberto, Great Neck, NY (US)

(21) Appl. No.: 12/312,735

(22) PCT Filed: Nov. 20, 2007
(86) PCT No.: PCT/US2007/024241

§ 371 Date: Nov. 18, 2010

Related U.S. Application Data
(60) Provisional application No. 60/860,599, filed on Nov. 22, 2006.

(51) Int. Cl. A01K 67/00; C12N 5/0735; G01N 33/00; C12Q 1/02

(52) U.S. Cl. 800/3; 800/12; 800/14; 435/325; 435/29; 800/21

(57) ABSTRACT

Provided are non-human mammals comprising a transgenic nucleic acid sequence capable of causing an alteration of expression of Bri2 or Bri3 in the mammal. Also provided are non-human mammals comprising a Bri2 or Bri3 gene under the control of the native Bri2 or Bri3 promoter. Additionally provided are non-human mammals genetically engineered to lack expression of a Bri2 or Bri3 gene. Further, non-human mammals comprising a transgene encoding a Bri2 or Bri3 protein under the control of the αCaMKII promoter are provided. Non-human mammals comprising a transgene encoding a furin protein are additionally provided. Embryonic stem cells of any of the above-described non-human mammals are further provided. Methods of screening a compound for treatment of a disease characterized by cerebral amyloidosis are additionally provided. Also provided are methods of making transgenic non-human mammals. Nucleic acids capable of causing an alteration of expression of Bri2 or BH3 if transfected into a mouse are additionally provided, as are comprising a sequence capable of causing an alteration of expression of Bri2 or Bri3 if transfected into a mouse.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of R21 AG027139-01 awarded by The National Institutes of Health.

### BACKGROUND OF THE INVENTION

1. Field of the Invention

The present invention generally relates to transgenic mammals. More specifically, the invention relates to transgenic mammals altered in expression of proteins involved in Alzheimer's Disease.

2. Description of the Related Art

Alzheimer's Disease (AD) is the most common cause of dementia in the world. It is estimated that ˜1% of humans aged 60-64 years have AD, increasing steadily to as many as 35%-40% after age 85 (Breteler et al., 1992). AD dementia progresses slowly leading to a severely impaired state, with behavioral symptoms, language dysfunction, incontinence, abnormal gait and complete social dependence. At autopsy, cerebral atrophy, neurofibrillary tangles and amyloid plaques are observed in the hippocampus, entorhinal cortex, amygdala and other areas. Neurofibrillary tangles are intraneuronal masses of abnormal, helically wound filaments that are composed of hyperphosphorylated forms of the tau protein. Amyloid plaques, also referred to as neuritic plaques, are deposits of extracellular fibrils of Aβ, a peptide derived from processing of the Amyloid Precursor Protein (APP), often surrounded by dystrophic dendrites and axons. Almost all AD cases present fibrillar Aβ deposits in cortical and/or meningeal micro vessels. In a minority of cases, this vascular amyloidosis, called congophilic amyloid angiopathy (CAA), is rather severe (Selkoe and Podlisny, 2002).

After advanced age, a positive family history of an AD-type dementia is the most important risk factor for AD (van Duijin et al., 1991). Genetic, epidemiological and clinical studies suggest a positive history of AD-like dementia in first-degree relatives in 40%-60% of cases. Moreover, 10%-15%, of all AD subjects has a family history consistent with an autosomal dominant trait. The latter cases are referred to as familial AD.

APP Gene Missense Mutations and AD. Clue to the localization of AD-causing genes came from the observation that trisomy 21 patients (Down's syndrome) develop AD already in early middle age, suggesting that a genetic defect causing AD would be localized to chromosome 21 (Glenner et al., 1984; Glenner and Wong, 1984) and that Down's syndrome patients develop Alzheimer pathology because of a gene dosage effect. In fact, when APP was cloned it was localized to chromosome 21q21.3-q22.05. The finding that a Down with translocation telomeric to the APP gene (Prasher et al., 1998) did not show AD symptoms and postmortem AD pathology at age 78, confirmed that the development of AD with plaques and tangles in trisomy 21 is due to APP over expression. Thus, early linkage studies in familial AD were focused on chromosome 21q. The discovery of a Val to Ile mutation within the transmembrane domain of APP and Carboxyl-terminal to the Aβ sequence in an English family was the first definitive implication of APP in early onset AD (EOAD) pathology (Goate et al., 1991). These discoveries were followed by description of other pathogenic mutations in other EOAD families and, as of today, 13 APP mutations that result in EOAD phenotype have been described (with or without CAA). Also, two more mutations have been described that result exclusively in a CAA phenotype (Selkoe and Podlisny, 2002).

APP is an ubiquitous type I transmembrane protein that undergoes a series of proteolytic events (Selkoe and Kopan, 2003; Sisodia and St George-Hyslop, 2002). APP is first cleaved at the plasma membrane or in intracellular organelles by β-secretase (Vassar et al., 1999). While the ectodomain is released extracellularly (sAPPβ) or into the lumen of intracellular compartments, the COOH-terminal fragment of 99 amino acids (C99) remains membrane bound. In a second, intramembraneous proteolytic event, C99 is cleaved, with somewhat lax site specificity, by the γ-secretase. Two peptides are released in a 1:1 stoichiometric ratio. The amyloidogenic Aβ peptide, consisting of 2 major species of 40 and 42 amino acids (Aβ40 and Aβ42, respectively) and an intracellular product named APP Intracellular Domain (AID) which is very short-lived and has been identified only recently (Passer et al., 2000; Cau and Sudhof, 2001; Cupers et al., 2001). In an alternative proteolytic pathway, APP is first processed by α-secretase in the Aβ sequence leading to the production of the soluble sAPPα ectodomain and the membrane bound COOH-terminal fragment of 83 amino acids (C83). C83 is also cleaved by the γ-secretase into the P3 and AID peptides. It is widely accepted that APP missense mutations cause AD by promoting the amyloidogenic processing pathway and generation of Aβ peptides (especially the Aβ42 form, considered to be more pathogenic than Aβ40) and the formation of amyloid fibrils (Selkoie and Podlisny, 2002).

PS1 and PS2 Gene Missense Mutations and AD. Mutations in APP explained only a small fraction of familial EOAD cases, indicating that inherited forms of AD were genetically heterogeneous (St George-Hyslop et al., 1990). Genome-wide linkage analyses suggested the presence of an EOAD locus on chromosome 14q (Schellenberg et al., 1993) and led to the identification of a novel gene, called Presenilin1 (Sherrington et al., 1995). Since it's discovery, 80 mutations that segregated with EOAD in a dominantly transmitted fashion have been identified. Shortly thereafter, a highly homologous gene on Ch1q31-42 called PS2, was discovered. PS2 is now known to be the site of 6 missense mutations causing familial EOAD (Rogaev et al., 1995; Levy-Lahad et al., 1995a; Levy-Lahad, 1995b). PS1 mutations cause the most aggressive forms of AD, and the affected patients are often symptomatic in the fifth decade of life and die in the sixth.

While the mechanistic connection between APP mutations and EOAD was obvious, the mechanism by which PS mutations lead to EOAD was puzzling. However, this puzzle was soon solved when it was found that PS are key components, together with Nicastrin, PEN2 and APH1, of a multi-molecular complex with γ-secretase activity (De Strooper et al., 1998; Yu et al., 2000; Francis et al., 2002; Goutte et al., 2002).

The importance of understanding the mechanisms regulating APP processing. Finding molecules that regulate APP processing without affecting the activity of either β- or γ-secretase in not only biologically relevant but is also of therapeutic interest. In fact, compounds targeting these molecules and capable of reducing the rate of APP cleavage would be specific (and effective) AD drugs. These compounds would be far superior to inhibitors of either β- or γ-secretase (two main targets for the development of AD drugs) because they would only interfere with APP processing. On the contrary, secretase inhibitors will interfere with cleavage of other substrates of secretases, therefore exerting toxic effects that may limit their therapeutic usefulness. This is a pressing problem since genetic and biochemical evidence indicates that secretase have many biologically important substrates. For example, γ-secretase also mediates the transmembranous cleavage of other membrane proteins including Notch, ErbB4, E-Cadherin, p75, APLP1, APLP2 and CD44 (DeStrooper et al., 1999; Ni et al., 2001; Marambaud et al., 2002; Marambaud et al., 2003; Scheinfeld et al., 2002; Lammich et al., 2002). Also, β-secretase contributes to myelination of peripheral nerves (Willem et al., 2006).

Is APP processing regulated by ligands? Cleavage of other γ-secretase substrates is regulated by ligands. Membrane-bound Notch, the best-studied γ-secretase substrate, is processed in 3 different sites. Notch is cleaved in the endoplasmic reticulum by Furin (cleavage occurs at the S1 site) and is expressed on the cell surface as a heterodimeric receptor. Interaction with ligands exposes the second cleavage site (S2) for proteolysis. Cleavage by the γ-secretase of the resultant C-terminal product, NEXT, releases the functionally active NICD. Since the similitude between APP and Notch signaling is striking, it is tempting to speculate that APP processing might also be regulated by specific ligands. Based on this analogy, we have postulated the existence of integral membrane proteins that can bind the ectodomain of APP and regulate its processing. These ligands might function at cell-cell contact sites (like for Notch) or might work in a cell-autonomous fashion. It is also conceivable that inhibitory ligands; i.e. ligands capable of interfering with or inhibiting APP processing, may exist. Finding APP ligand(s) will be instrumental to better understand the biological function of APP. In addition, ligands of APP would be ideal targets to develop therapeutic drugs. Compounds capable of mimicking ligands that inhibit APP processing or able to interfere with the interaction of APP with ligands that activate APP amyloidogenic cleavage would selectively reduce APP Aβ/AID production. These compounds would not affect the function of secretases and therefore will be voided of toxic effects associated with interfering with processing of other substrates of secretases.

Familial British And Danish Dementia. Familial British Dementia (FBD) and Familial Danish Dementia (FDD) are forms of autosomal dominant cerebral amyloidosis with extensive congophilic amyloid angiopathy (CAA). Clinically, progressive dementia, ataxia and spastic tetraparesis characterize FBD. In FDD patients, progressive dementia commences at the age of 40 and follows other earlier symptoms (Revesz et al., 2002).

BRI2 Gene Missense Mutations and FBD/FDD. Recently, mutations in BRI2, a gene located on chromosome 13 in humans, have been found in FBD (Vidal et al., 1999) and FDD (Vidal et al., 2000) patients. BRI2 codes for a Type II membrane proteins of unknown function. Both wild type and mutant BRI2 are processed by furin (Kim et al., 1999), resulting in the secretion of a C-terminal peptide. Furin cleavage of wild type BRI2 releases a 17 amino acid-long peptide. In FBD patients, a point mutation at the stop codon of BRI2 results in a read-through into the 3′-untranslated region and the synthesis of a BRI2 molecule containing 17 extra amino acids at the COOH-terminus. Furin cleavage generates a longer peptide, the ABri peptide, which is deposited as amyloid fibrils. In the Danish kindred, the presence of a 10-nt duplication one codon before the normal stop codon produces a frame-shift in the BRI2 sequence generating a larger-than-normal precursor protein, of which the amyloid subunit comprises the last 34 COOH-terminal amino acids.

Bri Proteins Interact with APP Inhibiting Aβ Production. Beside the effect of Bri proteins in FBD and FDD, those proteins bind APP and inhibit Aβ and AID production and β-secretase (PCT Patent Application No. PCT/US06/23135). The latter action inhibits sAPPβ production.

Based on the above, mouse models altered in BRI2, BRI3 or furin production would be useful for further determining the role of these three proteins in Alzheimer's and related diseases characterized by cerebral amyloidosis, and for screening for therapeutic compounds for the treatment of those diseases. Some work has been done in this regard. See Pickford et al., 2006. The present invention more fully addresses this need by taking novel approaches to the design and construction of transgenic mice altered in BRI production.

### SUMMARY OF THE INVENTION

Accordingly, transgenic mice were conceived and developed that are useful for studying Alzheimer's disease and related diseases, and for screening compounds for treatments for those diseases.

The invention is directed to non-human mammals comprising a transgenic nucleic acid sequence capable of causing an alteration of expression of Bri2 or Bri3 in the mammal. The mammals are made from models for Alzheimer's disease.

The invention is also directed to non-human mammals comprising a Bri2 or Bri3 gene under the control of the native Bri2 or Bri3 promoter. The Bri2 or Bri3 gene is one that does not naturally occur in the mammal.

Additionally, the invention is directed to non-human mammals genetically engineered to lack expression of a Bri2 or Bri3 gene.

The invention is also directed to non-human mammals comprising a transgene encoding a Bri2 or Bri3 protein under the control of the αCaMKII promoter.

Further, the invention is directed to non-human mammals comprising a transgene encoding a furin protein having an amino acid sequence at least 80% homologous to amino acids 108-794 of SEQ ID NO:3, wherein the non-human mammal is a model for Alzheimer's disease.

The invention is additionally directed to embryonic stem cells of any of the above-described non-human mammals.

The invention is further directed to somatic cells from any of the above mammals.

The invention is also directed to methods of screening a compound for treatment of a disease characterized by cerebral amyloidosis, dementia, and/or cognitive impairment. The methods comprise administering the compound to any one of the above-described mammals that has cerebral amyloidosis, dementia, and/or cognitive impairment, then determining whether the compound affects the cerebral amyloidosis, dementia, and/or cognitive impairment.

The invention is additionally directed to other methods of screening a compound for treatment of a disease characterized by cerebral amyloidosis, dementia, and/or cognitive impairment. These methods comprise administering the compound to a cell such as a neuron that has been isolated from one of the invention mammals that has cerebral amyloidosis, dementia, and/or cognitive impairment, then determining whether the compound affects ABri and/or Aβ-beta production, and/or the cerebral amyloidosis, dementia, and/or cognitive impairment.

The invention is further directed to methods of making a transgenic non-human mammal. The methods comprise
(a) transfecting embryonic stem cells of the mammal with a transgenic nucleic acid sequence capable of causing an alteration of expression of Bri2 or Bri3 in the mammal;
(b) injecting the transfected embryonic stem cells into blastocysts of the mammal and implanting the blastocysts into the uterus of a foster mother of the mammal;
(c) raising pups from the foster mother; and
(d) identifying a transgenic pup, which is the transgenic non-human mammal. In these methods, the mammal is a model of Alzheimer's disease.

The invention is also directed to other methods of making a transgenic non-human mammal. These methods comprise
(a) transfecting embryonic stem cells of the mammal with a transgenic nucleic acid sequence capable of causing an alteration of expression of Bri2 or Bri3 in the mammal;
(b) injecting the transfected embryonic stem cells into blastocysts of the mammal and implanting the blastocysts into the uterus of a foster mother of the mammal;
(c) raising pups from the foster mother; and
(d) identifying a transgenic pup, which is the transgenic non-human mammal. In these methods, the transgenic nucleic acid sequence comprises a Bri2 or Bri3 gene under the control of the αCaMKII promoter.

The invention is additionally directed to other methods of making a transgenic non-human mammal. The methods comprise
(a) transfecting embryonic stem cells of the mammal with a transgenic nucleic acid sequence capable of causing an alteration of expression of Bri2 or Bri3 in the mammal;
(b) injecting the transfected embryonic stem cells into blastocysts of the mammal and implanting the blastocysts into the uterus of a foster mother of the mammal;
(c) raising pups from the foster mother; and
(d) identifying a transgenic pup, which is the transgenic non-human mammal. In these methods, the transgenic non-human mammal does not express a Bri2 or Bri3.

Additionally, the invention is directed to nucleic acids capable of causing an alteration of expression of Bri2 or Bri3 if transfected into a mouse. These nucleic acids comprise a Bri2 or Bri3 gene under the control of the αCaMKII promoter.

The invention is further directed to nucleic acids comprising a sequence capable of causing an alteration of expression of Bri2 or Bri3 if transfected into a mouse. The sequence in these nucleic acids comprises a portion of a mouse genomic Bri2 or Bri3 gene such that the sequence could integrate into the mouse genome by homologous recombination to replace at least a portion of the native Bri2 or Bri3 gene.

### BRIEF DESCRIPTION OF THE DRAWINGS

FIG. 1 is a diagram and photographs relating to the generation of Bri2 transgenic mice. Panel A shows a schematic representation of the tgBRI2 construct. The fragments are not depicted on scale. The location of restriction enzymes and probe to be used on Southern blot analysis, as well as the PCR primers a and b is shown. Panel B shows the results of PCR of 18 pups (a total of 32 were tested). Three of the pups (1, 4 and 15) had integrated the BRI2 transgene. In the same PCR tube, β-actin was amplified to control for genomic DNA content (shown as “act.”). “Vec.” represents the control PCR performed using the transgenic vector as a template. Panel C shows a western blot of a wild type animal and the progeny of lines 1 and 4 with the αBRI2 antibody. The results indicate that the BRI2 protein is overexpressed in tgBRI2 animals.

FIG. 2 is a diagram and photographs relating to the generation of Bri2^{-/-} mice using a floxed BRI2 exon 2. Panel A is a schematic representation of the BRI2 gene locus, the targeting vector and the strategy to be used to generate BRI−/− and BRI2f/f mice. The black boxes represent the coding regions of the BRI2 exons. The location of restriction enzymes and probes to be used on Southern blot analysis, as well as the PCR primers (a, b, c and d) are also shown. Panel B shows PCR of seven of the 400 ES clones. Five of the 400 clones (only one, ES clone number 7, is shown here) had undergone homologous recombination of the BRI2 gene since the b-d and a-c PCRs amplify products of 1.9 and 2.4 Kb, respectively

FIG. 3 is diagrams and photographs relating to the generation and molecular characterization of ES cell clones carrying the human British or Danish mutations at one BRI2 allele. Panel A is diagrams showing the strategy and targeting vector for the generation of BRI2^{ADan/+} and BRI2^{ABri/+} mice. Panel B shows the results of PCR of ES clones (6 are shown here). Eight clones (3 for the Danish mutation, 5 for the British) had integrated the BRI2 transgene (for simplicity only positive clones BRI2^{ADan/+}344, BRI2^{ADan/+}339 and BRI2^{ABri/+}197 are shown here) as shown by the evidence that the b-d and a-c PCR amplifies products of 3.4 and 1.67 Kb, respectively. Panel C is a Southern blot of BamHI digested genomic DNA from a wild type and BRI2^{ADan/+344}, BRI2^{ADan/+339} and BRI2^{ABri/+197} ES clones. Hybridization with the 5′ probe visualizes a wild type 11.9 Kb band in the control clone (wt), while the BRI2^{ADan/I+}344, BRI2^{ADan/+}339 and BRI2^{ABri/+}197 ES clones present also the 8.9 Kb expected after homologous recombination.

FIG. 4 is graphs of ELISA determinations of Aβ40, Aβ42, sAPPα and sAPPβ from brains of CRND8 or littermate CRND8/BRI2tg animals. The Y axis is pg/ml for Aβ determinations and ng/ml for sAPPα and sAPPβ. The data show that BRI2 transgenic expression reduces the levels of all four APP-derived fragments.

FIG. 5 is micrographs of brain sections from CRND8 or littermate CRND8/BRI2tg animals immunohistochemistry stained of brains with an antibody against AD (monoclonal antibody 6E10). The figure shows reduced size and number of Aβ plaques in CRND8/BRI2tg animals compared to CRND8 littermates.

FIG. 6. Panel A is a schematic representation of the tgBRI2 construct. Note that the fragments are not depicted on scale. Refer to FIGS. 1A, 2A and 3A for more detailed descriptions. (B) See FIG. 1B description above. Panel C shows a western blot of two wild type animal including the progeny of lines BRI2-8.4, BRI2-8.5, BRI2 and ABri (these last two lines were obtained from Eileen MacGowan) with the aBRI2 antibody indicate that the BRI2 protein is over expressed in tgBRI2 animals. (D) Total brain sAPPα and sAPPβ were analyzed by ELISA at the indicated times (3, 4, an 6 mos). As shown for three independent BRI2 transgenic lines, BRI2 significantly reduces the levels of both α- and β-secretase-derived products. Collectively, the data in this figure demonstrates that BRI2 over expression inhibits APP processing in transgenic AD mice

FIG. 7. Panel A consists of cortical sections of 6 month old mice stained with αAβ 6E10. Panel B illustrates the quantification of amyloid plaque burden present in the brain of the indicated mouse groups. BRI2 transgene ameliorates significantly AD pathology of the CRND8 AD mice. The area occupied by amyloid plaques in the tgBRI2/CRND8 mice is expressed as a percentage of the amyloid area found in the CRND8 mice of the same experimental group, which is assumed to be 100%. This figure shows that BRI2 over expression reduces AD pathology in transgenic AD mice

FIG. 8. Panel A illustrates the Generation of Bri2−/− mice. See the detailed description in FIG. 2A. Panel B shows a PCR of 400 ES clones (for simplicity only seven clones are shown here) reveals that 5 of them (only one, ES clone number 7, is shown here) had undergone homologous recombination of the BRI2 gene since the b-d and a-c PCRs amplify products of 1.9 and 2.4 Kb, respectively. Panel C is a western blot analysis of brain membranes from Bri2+/+, Bri2+/− and Bri2−/− mice shows lack of or reduced levels of Bri2 expression in Bri2−/− and Bri2+/− mice, respectively. Calnexin is used as a control to verify equal loading of protein samples. Panel D is an analysis of brain membrane extracts from Bri2−/− and APP−/− mice. Total lysates were analyzed for Bri2 and APP expression (left panel). Brain lysates were immunoprecipitated with the αBRI2, αAPPct and rabbit polyclonal (RP) control antibody (right panel). Precipitates were analyzed for APP and Bri2 proteins. Bri2−/− and wild type mice express equal amounts of APP. Immunoprecipitation of endogenous APP with the αBRI2 antibody is specific since APP in precipitated wild type mice but neither APP−/− nor Bri2−/− mice. Panel E is a western blot analysis of brain membranes from Bri2+/+, Bri2+/− and Bri2−/− mice shows lack of or reduced levels of Bri2 expression in Bri2−/− and Bri2+/− mice, respectively. Calnexin is used as a control to verify equal loading of protein samples. Panel E represents Bri2+/− mice which were crossed to APP-PS1 tg AD mice to obtain Bri2+/−/APP-PS1 and Bri2+/+/APP-PS1 animals. Western Blot analysis of post nuclear supernatants of 8 month-old (2 Bri2+/−/APP-PS1 and 2 Bri2+/+/APP-PS1) and 4 month-old (1 Bri2+/−/APP-PS1 and 1 Bri2+/+/APP-PS1) shows the following: total APP (mouse plus transgenic human APP), C83, C99, Nct and human PS1DE9 levels are similar among mice in each age group. However, sAPPα is increased in the Bri2+/−/APP-PS1 mice as compared to the Bri2+/+/APP-PS1 littermates. As for total Aβ, the peptide is only detectable in 8 months old mice. Interestingly, total Aβ is increased in the two Bri2+/−/APP-PS1. Aβ is detected by two different monoclonal antibodies, 6E10 and 4G8. Panel F shows that ELISA detects Aβ40 and Aβ42 in all animals. The levels found in the Bri2+/−/APP-PS1 mice are expressed as a percentage of Aβ amounts in the Bri2+/+/APP-PS1, which is assumed to be 100%.

FIG. 9. Shows reference images of Aβ plaques of 6 month old mouse hippocampus. Five sections for each mouse genotype have been chosen to represent the vast populations of sections (over 1000 overall) analyzed. Different color contrasts represent the inter-experimental variability in DAB staining outcome, which does not interfere with the determination of the surface occupied by plaques, visible as dark brown areas. Artifacts in sections, when present, were manually corrected on ImageJ software, on the 8 bit/threshold image, according to the reference image (shown). (A-D), one series of 4 hippocampus sections, lateral to medial, 400 mm distant from each other (#17, 25, 33, 41). (E) “Bright” image of section in A: only the 6E10 stained areas are evident. F, 8 bit conversion of image in E, used for threshold process and for subsequent measurement, as detailed in G.

### DETAILED DESCRIPTION OF THE INVENTION

The present invention is directed to non-human mammals comprising a transgenic nucleic acid sequence capable of causing an alteration of expression of Bri2 or Bri3 in the mammal. The mammals are made from models for Alzheimer's disease.

As used herein, a “transgenic nucleic acid sequence” refers to an exogenous nucleic acid molecule that is introduced into the genome of a cell by artificial manipulations. The transgenic nucleic acid sequence may include nucleic acid sequences found in that animal so long as the introduced nucleic acid sequence contains some modification relative to the endogenous nucleic acid sequence (e.g., a point mutation, a deletion, the presence of a selectable marker gene, the presence of a loxP site, etc.) or is present in the genome where it does not occur naturally.

These mammals can comprise any transgenic nucleic acid sequence that can cause an alteration of expression of a Bri2 or Bri3 protein. As used herein, a Bri2 or Bri3 protein is a mammalian Type II membrane protein that has an amino acid sequence at least 70% homologous to SEQ ID NO:1 or SEQ ID NO:2, respectively.

The alteration in expression of the Bri2 or Bri3 protein can be, for example, an overexpression of the native or a human Bri2 or Bri3 sequence in the non-human mammal, or a knockout of the native Bri2 or Bri3 sequence in the mammal. Some of the transgenic nucleic acid sequences that can cause such an alteration comprise a segment that encodes at least a portion of a Bri2 or Bri3 protein. Preferably here, the portion of the Bri2 or Bri3 protein encodes at least a portion of a Bri2 or Bri3 protein that is at least 80% homologous to SEQ ID NO:1 or SEQ ID NO:2, respectively. More preferably, the portion of the Bri2 or Bri3 protein encodes at lease a portion of a Bri2 or Bri3 protein that is at least 90% homologous to SEQ ID NO:1 or SEQ ID NO:2. Even more preferably, the Bri2 or Bri3 protein is at least 99% homologous to SEQ ID NO:1 or SEQ ID NO:2, respectively. Most preferably, the transgenic nucleic acid sequence encodes at least a portion of a Bri2 or Bri3 protein that is a wild-type Bri2 or Bri3 protein. The wild-type Bri2 or Bri3 protein is most preferably a human protein.

Familial British Dementia (FBD) is characterized by a point mutation at the stop codon of BRI2, resulting in read-through into the 3′-untranslated region and the synthesis of a Bri2 protein containing 17 extra amino acids at the COOH-terminus. Furin cleavage generates a longer peptide, the ABri peptide, which is deposited as amyloid fibrils. In Familial Danish Dementia (FDD), the presence of a 10-nt duplication one codon before the normal stop codon produces a frame-shift in the BRI2 sequence generating a larger-than-normal precursor protein, of which the amyloid subunit comprises the last 34 COOH-terminal amino acids. Transgenic nucleic acid sequences encoding the FBD and FDD Bri2 proteins in non-human mammals are envisioned as within the scope of the present invention.

Thus, in some of the invention mammals, the transgenic nucleic acid segment comprises a Bri2 gene with a mutation in the stop codon allowing translational read-through as with a human Bri2 gene associated with Familial British Dementia (FBD). Preferably, the segment encodes a human Bri2 protein associated with Familial British Dementia (FBD).

In other mammals of the present invention, the segment comprises a Bri2 gene with a decamer duplication in the 3′ region as with the human gene associated with Familial Danish Dementia (FDD). Preferably, the segment encodes a human Bri2 protein associated with FDD.

To be able to further characterize the role of the Bri2 and Bri3 proteins in Alzheimer's disease and normal physiology, it is envisioned that the mammals of the instant invention include those where the function of the Bri2 or Bri3 protein is eliminated. One way to obtain such mammals is by inserting the transgenic nucleic acid sequence into the native BRI2 or BRI3 gene, or replacing a portion of the native BRI2 or BRI3 gene with a transgenic nucleic acid sequence that precludes production of the functional Bri2 or Bri3 protein.

Accordingly, the mammals of the present invention include those where the transgenic nucleic acid sequence is an insert into, or a replacement of, at least a portion of a native Bri2 or Bri3 gene. The insert in these mammals are not limited to any particular insertion or replacement; there are a multitude of potential insertions or replacements that would be useful, particularly for eliminating function of the native protein. Preferably, the insert or replacement deletes the native BRI2 exon 2 (see Example).

The non-human mammal can be any Alzheimer's Disease (AD) model now known or later discovered. Preferred mammals are rats and mice, most preferably mice. Preferred mouse models of AD produce a human APP protein. Most of those are generated by transgenic overexpression of human pathogenic APP mutants, alone or in combination with human PS pathogenic mutants.

Non-limiting examples of mice AD models include B6.129-Psen1^{tm1Mpm}/J; B6.129S2-Tg(APP)8.9Btla/J; B6.Cg-Tg(APPswe,PSEN1dE9)85 Dbo/J; B6.Cg-Tg(PDGFB-APP)5Lms/J; B6.Cg-Tg(PDGFB-APPSwlnd)20Lms/1J; B6.Cg-Tg(PDGFB-APPSwlnd)20Lms/2J; B6C3-Tg(APPswe,PSEN1dE9)85 Dbo/J; B6.Cg-Tg(APP695)3Dbo; Tg(PSEN1 dE9)S9 Dbo/J; C3B6-Tg(APP695)3Dbo/J Mapttm1 (EGFP)Klt Tg(MAPT)8cPdav/J and B6.Cg-Mapttm1(EGFP)KitTg(MAPT)8cPdav/J (all available at Jackson Laboratory, Bar Harbor, Me.), and mThy1-hAPPtm.

Most preferably, the mammal is a TgCRND8 mouse (see Example). The TgCRND8 mouse is one of the better-characterized AD models and expresses a mutant (K670N/M671L and V717F) human APP transgene under the regulation of the Syrian hamster prion promoter on a C3H/B6 strain background (Janus et al., 2000). These mice present spatial learning deficits at 3 months of age that are accompanied by both increasing levels of SDS-soluble Aβ and increasing numbers of Aβ-containing amyloid plaques in the brain (Janus et al., 2000).

The alteration of expression of Bri2 or Bri3 in the invention mammals can be conditional. Conditional gene inactivation provides a means to control the development and tissue-specificity of gene disruption Where the alteration of expression is conditional, that conditional alteration of expression can be achieved by flanking the sequence with a loxP site (sometimes called a “foxed” sequence) in a mammal where a Cre recombinase can be conditionally expressed. As is known, when the Cre recombinase is expressed, the foxed sequence will be deleted. Thus, the alteration of expression is achieved in this system when the Cre recombinase is expressed.

Where the transgenic nucleic acid sequence is inserted into, or replaces at least a portion of the native Bri gene, the sequence can comprise a non-Bri sequence, causing a knockout of the Bri gene. Here, the non-Bri sequence is preferably a selectable marker so that the insertion can be selected for. A preferred selectable marker is PGK-neo, which contains a neomycin-resistance gene under the control of the PGK promoter (see Example).

Where the transgenic nucleic acid sequence comprises a segment that encodes at least a portion of the Bri2 or Bri3 protein, the sequence preferably further comprises a promoter that directs expression of the Bri2 or Bri3 protein to the brain of the mammal. Any promoter known in the art can be used here. The selection of a promoter that directs expression of the Bri2 or Bri3 can be chosen by the skilled artisan without undue experimentation. In order to provide for Bri2 or Bri3 expression most relevant to disorders characterized by cerebral amyloidosis, the promoter most preferably directs expression of the Bri2 or Bri3 protein to the forebrain of the mammal. A preferred example of such a promoter is the αCaMKII promoter (see Example).

Where the transgenic nucleic acid sequence comprises a segment that encodes at least a portion of the Bri2 or Bri3 protein, the Bri2 or Bri3 protein can be expressed constitutively in the adult of the mammal. Alternatively, the Bri2 or Bri3 can be inducible in the adult of the mammal.

Preferably, the mammals of the invention are mice, and the transgenic nucleic acid sequence comprises a segment encoding an αCaMKII promoter operably liked to a Bri2 gene. More preferably, the Bri2 gene is overexpressed in the postnatal forebrain of the mammal. Also, it is also preferred that the Bri2 gene encodes a human Bri2 protein. In some of these mice, the human Bri2 protein is preferably at least 98% homologous to SEQ ID NO:1. In others, the Bri2 gene preferably comprises a mutation in the stop codon allowing translational read-through as with a human Bri2 gene associated with Familial British Dementia (FBD). Most preferably, the Bri2 gene encodes a human Bri2 protein associated with Familial British Dementia (FBD).

In other invention mice, the transgenic nucleic acid sequence comprises a segment encoding an αCaMKII promoter operably liked to a Bri2 gene, where the Bri2 gene comprises a decamer duplication in the 3′ region as with the human gene associated with Familial Danish Dementia (FDD). Preferably, the Bri2 gene encodes a human Bri2 protein associated with FDD.

In additional invention mice, the transgenic nucleic acid sequence comprises a LoxP site such that exon 2 of the Bri2 gene is deleted upon induction of Cre-mediated recombination.

Other preferred invention mammals are mice, and the transgenic nucleic acid sequence comprises a Bri2 exon 6 homologously inserted into the mouse Bri2 gene, where the Bri2 exon 6 comprises a mutation in the stop codon allowing translational read-through as with a human Bri2 gene associated with Familial British Dementia (FBD).

Additional preferred invention mammals are mice, and the transgenic nucleic acid sequence comprises a Bri2 exon 6 homologously inserted into the mouse Bri2 gene, where the Bri2 exon 6 comprises a decamer duplication as with the human gene associated with Familial Danish Dementia (FDD).

The invention is also directed to non-human mammals comprising a Bri2 or Bri3 gene under the control of the native Bri2 or Bri3 promoter. The Bri2 or Bri3 gene is one that does not naturally occur in the mammal. Preferably, the mammal is a mouse. It is also preferred if the Bri2 or Bri3 gene is a human Bri2 or Bri3 gene. In some of these mice, the Bri2 or Bri3 gene is a Bri2 gene comprising a mutation in the stop codon allowing translational read-through as with a human Bri2 gene associated with Familial British Dementia (FBD). Preferably, the Bri2 gene encodes a human Bri2 protein associated with Familial British Dementia (FBD). In others of these mice, the Bri2 or Bri3 gene is a Bri2 gene comprising a decamer duplication in the 3′ region as with the human gene associated with Familial Danish Dementia (FDD). Preferably, the Bri2 gene encodes a human Bri2 protein associated with FDD. Most preferably, the mammal is a model for Alzheimer's disease.

Additionally, the invention is directed to non-human mammals genetically engineered to lack expression of a Bri2 or Bri3 gene. Preferably, the mammal is a mouse. It is also preferred that the mammal is a model for Alzheimer's disease. Some of these mice lack expression of a Bri2 gene. Others lack expression of a Bri3 gene.

The invention is also directed to non-human mammals comprising a transgene encoding a Bri2 or Bri3 protein under the control of the αCaMKII promoter. Preferably, the mammal is a mouse. It is also preferred that the mammal is a model for Alzheimer's disease. Most preferably, the Bri2 or Bri3 protein is a human protein.

Further, the invention is directed to non-human mammals comprising a transgene encoding a furin protein having an amino acid sequence at least 80% homologous to amino acids 108-794 of SEQ ID NO:3. Some of these mammals are models for Alzheimer's disease; others are not models for Alzheimer's disease. Such mammals are useful for various purposes, for example studying the physiology of Alzheimer's disease and screening for Alzheimer's disease treatments. Preferably, the mammal is a mouse. The furin protein preferably has an amino acid sequence at least 90% homologous to amino acids 108-794 of SEQ ID NO:3. More preferably, the furin protein has an amino acid sequence at least 95% homologous to amino acids 108-794 of SEQ ID NO:3. Most preferably, the furin protein is a human furin protein. The furin protein is can be a knock-in alteration of a homologous furin protein.

With any of the invention mammals, the invention encompasses mammals that are heterozygous for the transgenic haplotype. The invention also encompasses mammals that are homozygous for the transgenic haplotype.

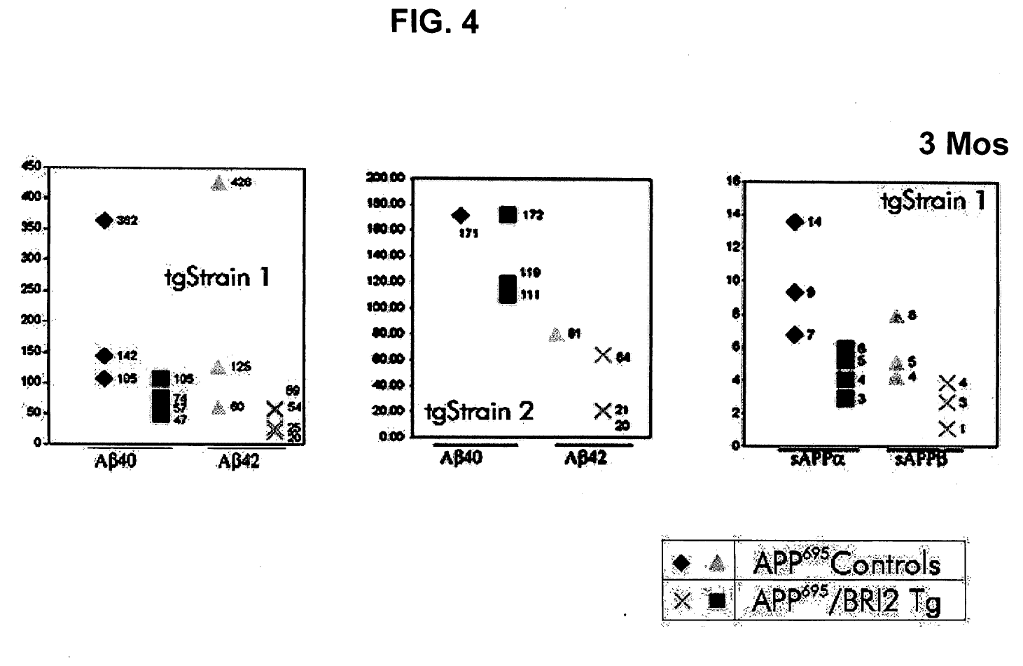

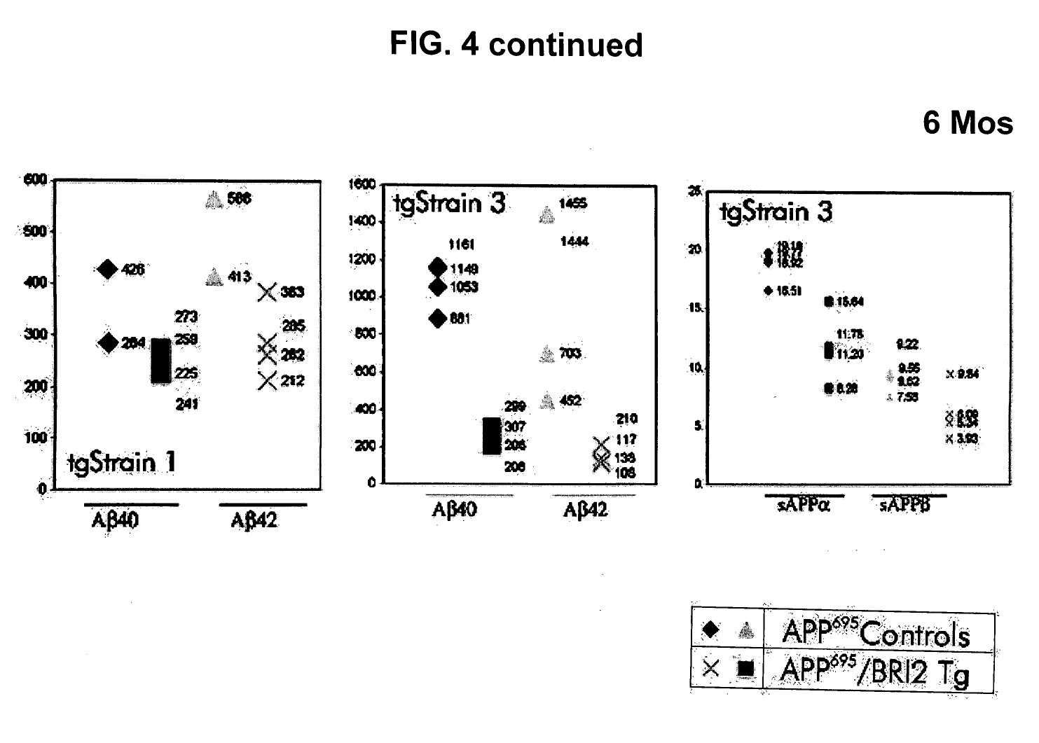

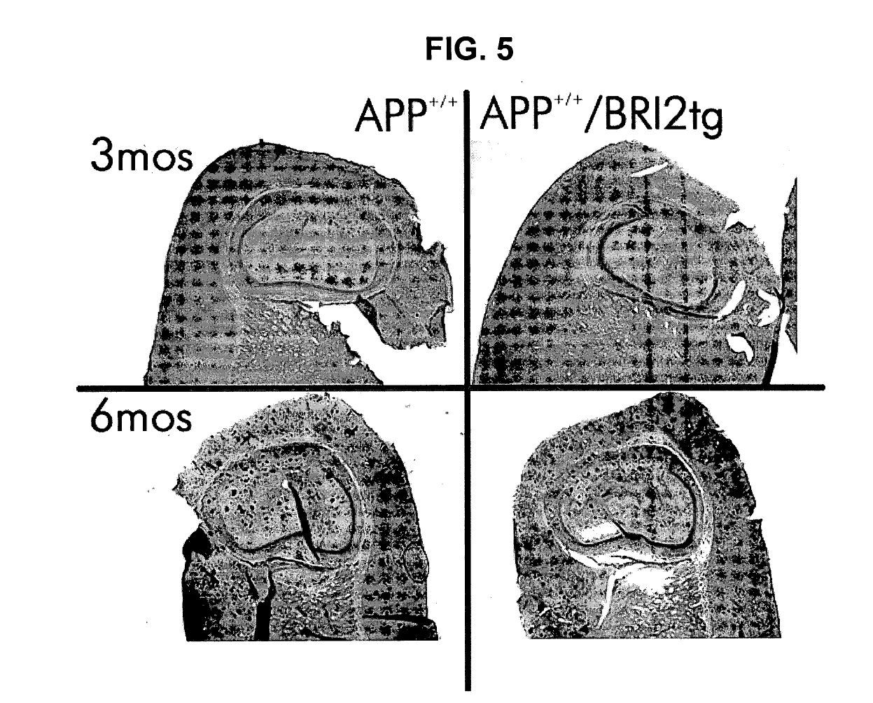

The invention mammals that have reduced amyloidosis (see, e.g., FIGS. 4 and 5) preferably show an enhanced cognitive ability over the mammal without the transgenic nucleic acid sequence. Preferred cognitive abilities here include novel object recognition, reference memory, special working memory, fear conditioning, or learning and memory. These can be evaluated without undue experimentation. See, e.g., the various tests described in http://www.psychogenics.com. Further, the invention mammals that have increased amyloidosis preferably show a decreased cognitive ability over the mammal without the transgenic nucleic acid sequence.

The invention is additionally directed to embryonic stem (ES) cells of any of the above-described non-human mammals.

The invention is further directed to somatic cells from any of the above mammals. These somatic cells can be primary cells or cells that can be stably maintained in culture. These can be any somatic cells from the mammals, including adult stem cells, epithelial cells, connective tissue cells, or, preferably, nervous tissue cells. More preferably, the somatic cell is a neuron, most preferably a glial cell or an astrocyte.

The invention is also directed to methods of screening a compound for treatment of a disease characterized by cerebral amyloidosis, dementia, and/or cognitive impairment. The methods comprise administering the compound to any one of the above-described invention mammals that has cerebral amyloidosis, dementia, and/or cognitive impairment, then determining whether the compound affects the cerebral amyloidosis, dementia, and/or cognitive impairment. Depending on the invention mammal used, the disease is preferably Alzheimer's disease, Familial British Dementia, or Familial Danish Dementia. The most appropriate invention mammal for these screening methods can be determined by the skilled artisan without undue experimentation. In these screening methods, the mammal is preferably a mouse.

For some of these methods, determining whether the compound affects the cerebral amyloidosis, dementia, and/or cognitive impairment is performed by determining whether the compound increases a cognitive ability of the mammal. Preferred cognitive abilities that can be determined here are novel object recognition, reference memory, special working memory, fear conditioning, or learning and memory. Preferably, the disease here is Alzheimer's disease. A preferred mammal is one of the above-described invention transgenic mice that shows an enhanced cognitive ability over the mammal without the transgenic nucleic acid sequence.

These screening methods are not limited to any particular compound. The compound can be, for example, a oligopeptide or a protein. Where the compound is an oligopeptide or a protein, it can comprise an antigen binding site of an immunoglobulin. The compound can also be a nucleic acid, e.g., an miRNA, a ribozyme or an aptamer. Most preferably, the compound is an organic molecule less than 2000 MW.

The invention is additionally directed to other methods of screening a compound for treatment of a disease characterized by cerebral amyloidosis, dementia, and/or cognitive impairment. These methods comprise administering the compound to a cell such as a neuron isolated from one of the invention mammals that has cerebral amyloidosis, dementia, and/or cognitive impairment, then determining whether the compound affects ABri or Aβ production, or the cerebral amyloidosis, dementia, and/or cognitive impairment ability. In some of these methods, ABri production is determined. In other of these methods, ADAN is determined. In still other of these methods, Aβ production is determined. Cognitive assessment may be made directly on the mammals cognitive ability through tests described above. Here, Aβ can be determined indirectly, e.g., by measuring changes in α-, β-, or γ-secretase activity, or production of sAPPβ, or AID.

In some of these methods, the neuron is from a mammal comprising a transgenic Bri2 gene with a mutation in the stop codon allowing translational read-through as with a human Bri2 gene associated with Familial British Dementia (FBD). Most preferably, the transgenic Bri2 gene encodes a human Bri2 protein associated with Familial British Dementia (FBD). In other of these methods, the neuron is from a mammal comprising a transgenic Bri2 gene with a decamer duplication in the 3′ region as with the human gene associated with Familial Danish Dementia (FDD). In other of these methods, the neuron is from a mammal lacking the Bri2 gene, or lacking the fully functional Bri2 gene and/or protein. Most preferably, the transgenic Bri2 gene encodes a human Bri2 protein associated with FDD.

The invention is further directed to methods of making a transgenic non-human mammal. The methods comprise, first, transfecting embryonic stem cells of the mammal with a transgenic nucleic acid sequence capable of causing an alteration of expression of Bri2 or Bri3 in the mammal; then injecting the transfected embryonic stem cells into blastocysts of the mammal and implanting the blastocysts into the uterus of a foster mother of the mammal; third, raising pups from the foster mother; and identifying a transgenic pup, which is the transgenic non-human mammal. In these methods, the mammal is a model of Alzheimer's disease. Preferably, the mammal is a mouse.

Each step of these methods is preferably monitored, e.g., by restriction digestion and Southern blotting; polymerase chain reaction (PCR) and/or sequencing, as appropriate, to determine the presence, location, ploidy level, copy number, whether the insert was by homologous recombination, and/or structure of the transgenic nucleic acid sequence in the ES cells and/or the pups; ELISA, western blotting and/or RT-PCR to determine expression of genes that are in the transgenic nucleic acid sequence; etc.

In some of these transgenic mammals, the Bri2 or Bri3 is not expressed. In others, the Bri2 or Bri3 is expressed. Where the Bri2 or Bri3 is expressed, the transgenic non-human mammal preferably expresses a human Bri2 or Bri3.

In some of these methods, the transgenic nucleic acid sequence comprises a Bri2 gene comprising a mutation in the stop codon allowing translational read-through as with a human Bri2 gene associated with Familial British Dementia (FBD). Here, the Bri2 gene preferably encodes a human Bri2 protein associated with Familial British Dementia (FBD). In others of these methods, the transgenic nucleic acid sequence comprises a Bri2 gene comprising a decamer duplication in the 3′ region as with the human gene associated with Familial Danish Dementia (FDD). Here, the Bri2 gene preferably encodes a human Bri2 protein associated with FDD.

Production of the FBD or FDD mammals discussed above preferably uses a knock in (KI) approach, where the FBD or FDD gene is inserted into the genome by homologous recombination. The KI approach is preferred since it allows faithful and precise reproduction of the genetic defect associated with FBD and FDD.

Additionally, the invention is directed to methods of making a transgenic non-human mammal. These methods comprise first, transfecting embryonic stem cells of the mammal with a transgenic nucleic acid sequence capable of causing an alteration of expression of Bri2 or Bri3 in the mammal; then injecting the transfected embryonic stem cells into blastocysts of the mammal and implanting the blastocysts into the uterus of a foster mother of the mammal; third, raising pups from the foster mother; and identifying a transgenic pup, which is the transgenic non-human mammal. In these methods, the transgenic nucleic acid sequence comprises a Bri2 or Bri3 gene under the control of the αCaMKII promoter.

The invention is also directed to methods of making a transgenic non-human mammal. The methods comprise first, transfecting embryonic stem cells of the mammal with a transgenic nucleic acid sequence capable of causing an alteration of expression of Bri2 or Bri3 in the mammal; then injecting the transfected embryonic stem cells into blastocysts of the mammal and implanting the blastocysts into the uterus of a foster mother of the mammal; third, raising pups from the foster mother; and identifying a transgenic pup, which is the transgenic non-human mammal. In these methods, the transgenic non-human mammal does not express a Bri2 or Bri3.

The invention is additionally directed to other methods of making a transgenic non-human mammal. The methods comprise first, transfecting embryonic stem cells of the mammal with a transgenic nucleic acid sequence capable of causing an alteration of expression of furin in the mammal; then injecting the transfected embryonic stem cells into blastocysts of the mammal and implanting the blastocysts into the uterus of a foster mother of the mammal; third, raising pups from the foster mother; and identifying a transgenic pup, which is the transgenic non-human mammal. Preferably, the mammal is a model of Alzheimer's disease. It is also preferred that that the mammal is a mouse. Preferably, the transgenic nucleic acid sequence comprises at least a portion of a furin gene. That furin gene preferably encodes a furin protein has an amino acid sequence at least 95% homologous to amino acids 108-794 of SEQ ID NO:3. Most preferably, the furin gene is a human furin gene.

Also, the invention is directed to nucleic acids capable of causing an alteration of expression of Bri2 or Bri3 if transfected into a mouse. The nucleic acids comprise a Bri2 or Bri3 gene under the control of the αCaMKII promoter.

Further, the invention is directed to nucleic acids comprising a sequence capable of causing an alteration of expression of Bri2 or Bri3 if transfected into a mouse. The sequence comprises a portion of a mouse genomic Bri2 or Bri3 gene such that the sequence could integrate into the mouse genome by homologous recombination to replace at least a portion of the native Bri2 or Bri3 gene. With some of these nucleic acids, a mouse transfected with the nucleic acid does not express the Bri2 or Bri3 protein. With other of these nucleic acids, the sequence comprises a Bri2 gene comprising a mutation in the stop codon allowing translational read-through as with a human Bri2 gene associated with Familial British Dementia (FBD). Here, the Bri2 gene encodes a human Bri2 protein associated with Familial British Dementia (FBD). With still other of these nucleic acids, the sequence comprises a Bri2 gene comprising a decamer duplication in the 3′ region as with the human gene associated with Familial Danish Dementia (FDD). Here, the Bri2 gene encodes a human Bri2 protein associated with FDD. Still other of these nucleic acids further comprise a loxP site. Preferably, the loxP site is in the genomic Bri2 or Bri3 gene. It is also preferred that these nucleic acids further comprise a floxed PGK-neo positive selection cassette. See, e.g., the Example. Additionally preferred nucleic acids here further comprise a PGK-dt negative selection cassette. See, e.g., the Example. The most preferred nucleic acids comprise a loxP site in a genomic Bri2 gene, a floxed PGK-neo positive selection cassette, and a PGK-dt negative selection cassette.

Preferred embodiments of the invention are described in the following Example. Other embodiments within the scope of the claims herein will be apparent to one skilled in the art from consideration of the specification or practice of the invention as disclosed herein. It is intended that the specification, together with the Example, be considered exemplary only, with the scope and spirit of the invention being indicated by the claims, which follow the examples.

### Example

### Generation of BRI2-Mutant Mice

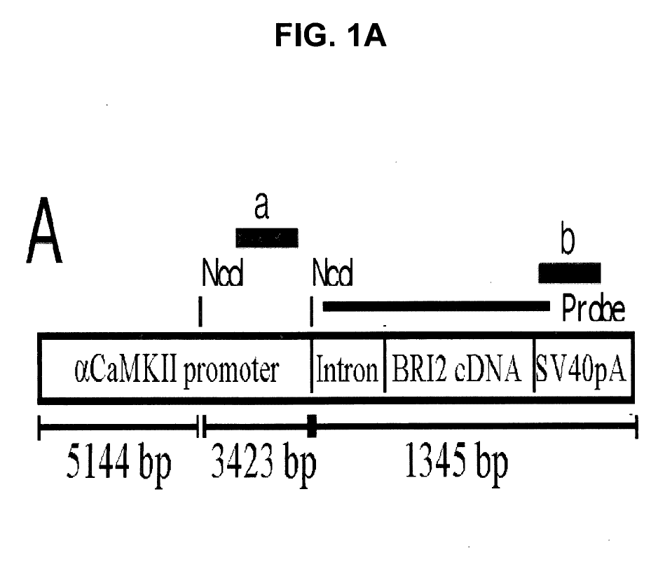

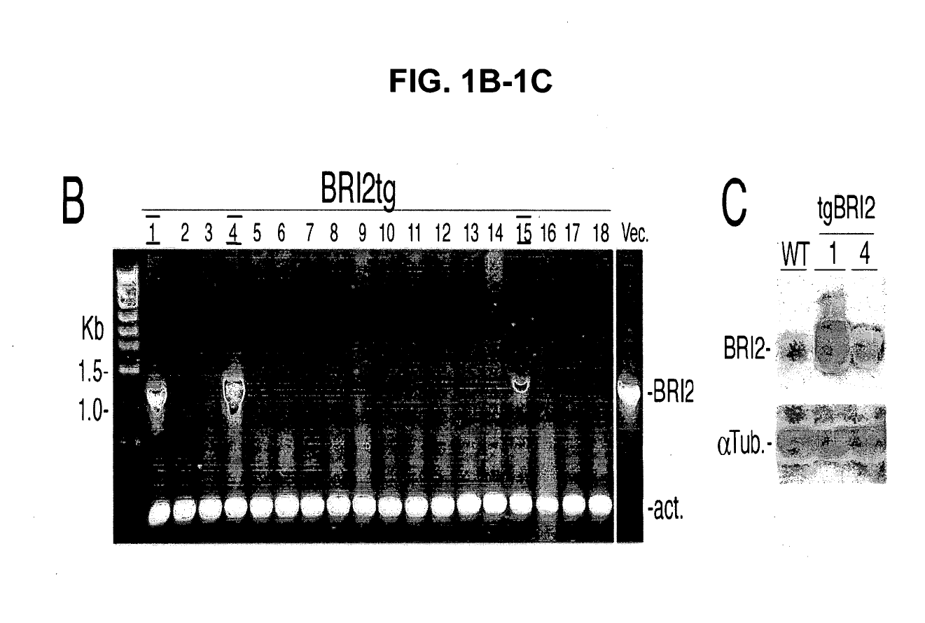

Generation of BRI2 transgenic mice. Using an approach similar to that described previously (Zazzeroni et al., 2003), an αCaMKII-BRI2 transgene was constructed. In this construct, the αCaMKII promoter segment contains ˜8.5 Kb genomic DNA upstream of the transcription initiation site of the αCaMKII gene and 84 bp of the 5′ gene noncoding exon, which is followed by a hybrid intron, BRI2 cDNA, and the SV40 polyadenylation signal (FIG. 1A). Thus, this promoter-enhancer region drives transcription of BRI2 in the postnatal forebrain, which is the area markedly affected by AD. This selective spatiotemporal expression will avoid issues arising from expression of the transgene during development or in other organs and tissues.

The above construct was injected into pronuclei of FVB embryos. Thirty-two pups were genotyped for the presence of the transgene by polymerase chain reaction (PCR) on tail DNA using primers a and b (indicated in FIG. 1A). Three founders were found (FIG. 1B-tgBRI2-1, tgBRI2-4 and tgBRI2-15). The founder animals were mated with FVB mice and germline transmission was observed in two lines (tgBRI2-1 and tgBRI2-4). The expression levels of BRI2 transgene in the brains of transgenic lines were determined by western blot analyses using the αBRI2 antibody. Since this antibody cross-reacts with both human and mouse BRI2 protein, the BRI2 expression found in the tgBRI2 animals was compared to that of wild type littermates. As shown in FIG. 1C, the BRI2 protein levels in the two lines (tgBRI2-1 and tgBRI2-4) appear to be ˜10 (tgBRI2-1) and ˜2 (tgBRI2-4)-fold that of wild type animals (a-tubulin was used as an internal standard to normalize for protein loading).

Generation and molecular characterization of ES cell clones carrying a floxed BRI2 exon 2. BRI2-null mice provide an excellent animal model to study the role of BRI2 in APP processing as well as AD pathogenesis and progression. BRI2 exon 2 was deleted because it contains the transmembrane region and the proximal part of the extracellular region of BRI2, which is involved in APP interaction. The mRNA transcribed by this locus after exon 2 deletion has the potential of producing a BRI2 polypeptide containing part of the BRI2 cytoplasmic tail fused to part of the extracellular region of BRI2. This polypeptide, if formed, will lack the transmembrane region and will not be integrated in cell membranes, where it associates with APP. Thus, even if such a BRI2 polypeptide is generated, it will not interact with APP and will not interfere with APP processing.

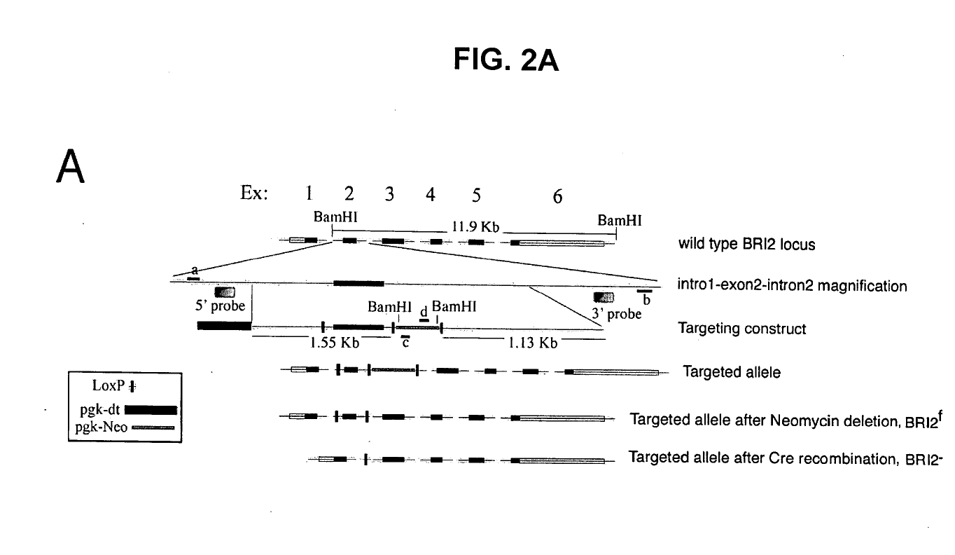

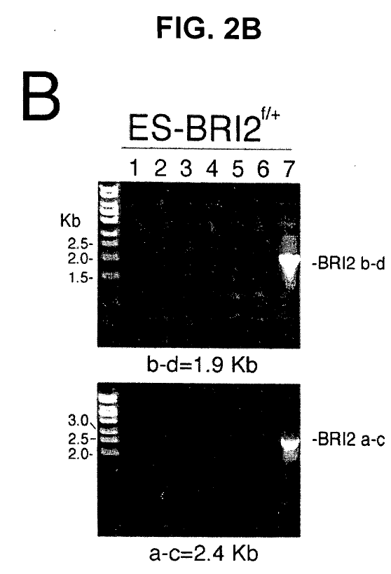

Based on information derived by analysis of the mouse genome sequence a targeting vector was constructed in which a loxP site is placed in the genomic BRI2 sequence ˜200 bp 5′ of exon 2. A floxed positive selection cassette, PGK-neo, which contains a neomycin-resistance gene under the control of the PGK promoter, was inserted into intron 2 of the BRI2 gene, ˜200 bp 3′ of exon 2. The rationale for the use of the floxed PGK-neo positive selection cassette is the ability to remove the selection cassette by Cre-mediated recombination, eliminating the possibility that presence of the cassette might affect expression of the targeted locus or neighboring genes. To complete the targeting vector, a negative selection cassette, PGK-dt, which encodes the diphtheria toxin, was included to enrich for ES cell clones carrying the correct homologous recombination events. Schematic of the constructs and of the strategy are shown in FIG. 2A.

The linearized targeting vector was transfected into 129 ES cells by electroporation. In the presence of the positive selection drug G418, only those clones in which the PGK-neo selection cassette has been integrated and the PGK-dt cassette has been removed by homologous recombination survive. ES cell clones carrying the targeting vector by random, non-homologous integration are eliminated due to expression of diphtheria toxin. After selection in G418-containing medium, 400 ES clones were picked and expanded. Genomic DNA from each clone was prepared and screened for the correct homologous recombination events in both 5′ and 3′ homologous regions by PCR using the primer couples a-c (5′ region) and b-d (3′ region). The schematic localization of these primers is shown in FIG. 2A. These primers amplify fragments of the expected sizes (2.4 and 1.9 Kb, respectively) only if homologous recombination has occurred. One primer (c for the 5′ region and d for the 3′ end) is in the PGK-neo selection cassette while the other (a and b for the 5′ and 3′ regions, respectively) is in the genomic BRI2 region outside the targeting vector. Out of 400 ES clones screened, 5 clones had integrated the loxP sites and PGK-neo in one of the endogenous BRI2 alleles (a representative sample is shown in FIG. 2B). The occurrence of homologous recombination was also verified for each clone by sequencing the PCR products (not shown). This analysis has also confirmed the insertion of the loxP sites.

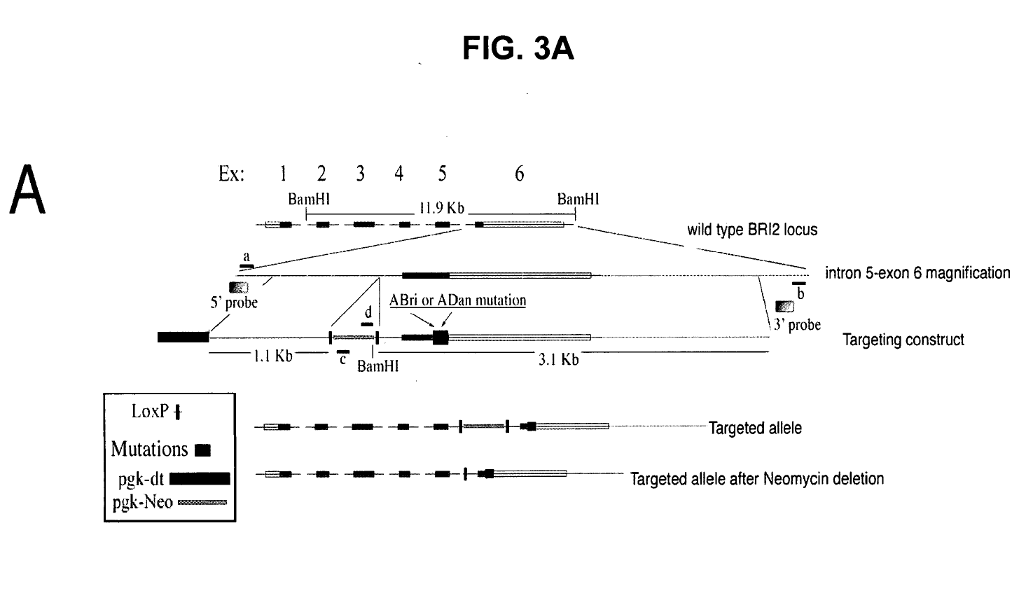

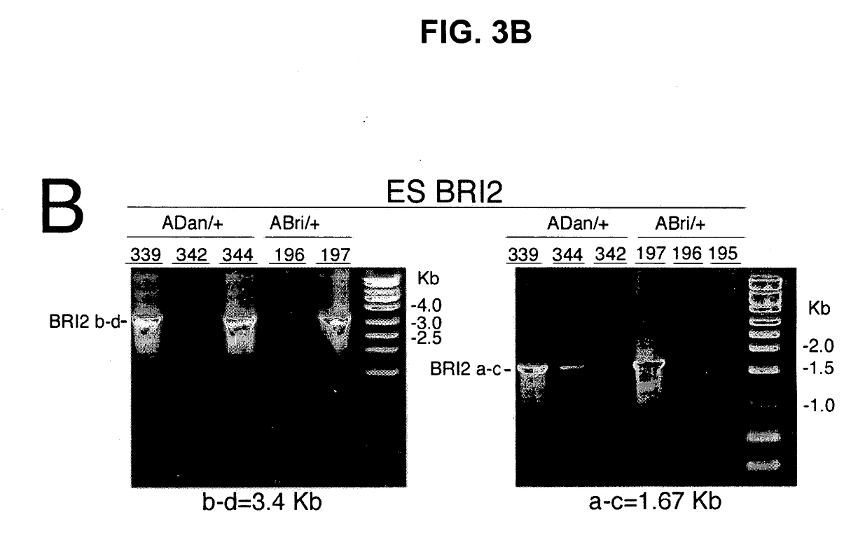

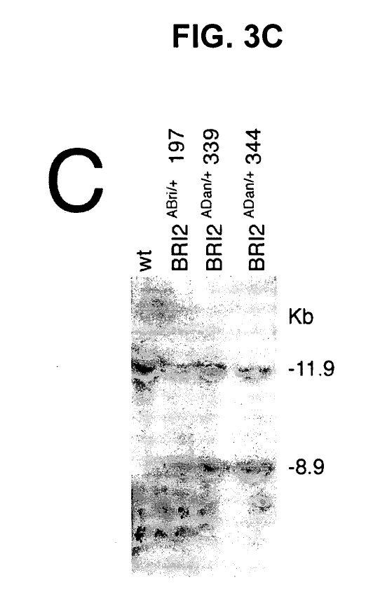

Generation and molecular characterization of ES cell clones carrying either the human British or Danish mutations at one BRI2 allele. The targeting strategy for the generation of the mutant BRI2 KI mice entails the replacement of the BRI2 exon 6 with mutated exon 6 carrying either the FDD or the FBD mutations. Two targeting vectors were generated for the introduction of FBD and FDD BRI2 mutations. The targeting vectors used the floxed PGK-neo selection cassette and contained the same 5′ homologous region and the negative selection cassette, PGK-dt as the vectors described above (FIG. 3A). The 3′ homologous region is vector-specific and introduces the FBD or the FDD mutations and a BamHI site into the BRI2 mouse gene (FIG. 3A).

The linearized KI targeting vectors for the introduction of the FBD and FDD mutations were transfected into 129 ES cells by electroporation and selected as described above. ES cell clones carrying the proper homologous recombination were identified by PCR using primers a-c for the 5′ region (if homologous recombination has occurred these primers will amplify a product of 1.67 Kb) and primers b-d for the 3′ region (amplified fragment from ES clones undergone homologous recombination is of 3.4 Kb). Primers c and d are the same as those shown in FIG. 2A. From the ˜600 ES clones analyzed three clones were found in which the Danish mutation was inserted in one of the BRI2 alleles and five in which the British mutation was knocked in. In FIG. 3B, representative positive (BRI2^{ADan/+}344, BRI2^{ADan/+}339 and BRI2^{ABri/+}197) and negative (BRI2^{ADan/+}342, BRI2^{ABri/+}195 and BRI2^{ABri/+}196) ES clones are shown. The occurrence of homologous recombination was confirmed by sequencing the PCR products (not shown).

Proper homologous recombination in ES cell clones was confirmed by performing Southern blot analysis. Homologous recombination was verified at the 5′ homologous region (FIG. 3C). In this experiment, genomic DNA derived from individual BRI2^{ABri/+} and BRI2^{ADan/30} ES clones was digested with BamHI, gel separated, blotted into a nylon membrane and hybridized with the 5′ probe (see FIG. 3A). This probe hybridizes with a ˜11.9 Kb fragment derived from the wild-type locus (FIG. 3A). If the desired recombination events have occurred, the 5′ probe yields a ˜8.9 Kb fragment upon BamHI digestion due to the introduction of the BamHI site and the PGK-neo selection cassette (FIG. 3A). As shown in FIG. 3C for three representative clones (BRI2^{ADan/+}344, BRI2^{ADan/+}339 and BRI2^{ABri/+}197), these ES clones carry a wild type allele (11.9 Kb) and a recombined allele (8.9 Kb). Of note, the 11.9 Kb and 8.9 Kb bands have similar intensity, showing that 50% of the BRI2 alleles are wild type and 50% are recombined. This proves that the selected ES cells are clonal populations.

Production of Aβ40, Aβ42, sAPPα and sAPPβ was determined in brains of CRND8 or littermate CRND8/BRI2tg animals using an IBL (Minneapolis Minn.) kit. Absorbance at A₄₅₀ was measured. Results are shown in FIG. 4. The data show that BRI2 transgenic expression reduces the levels of all four APP-derived fragments.

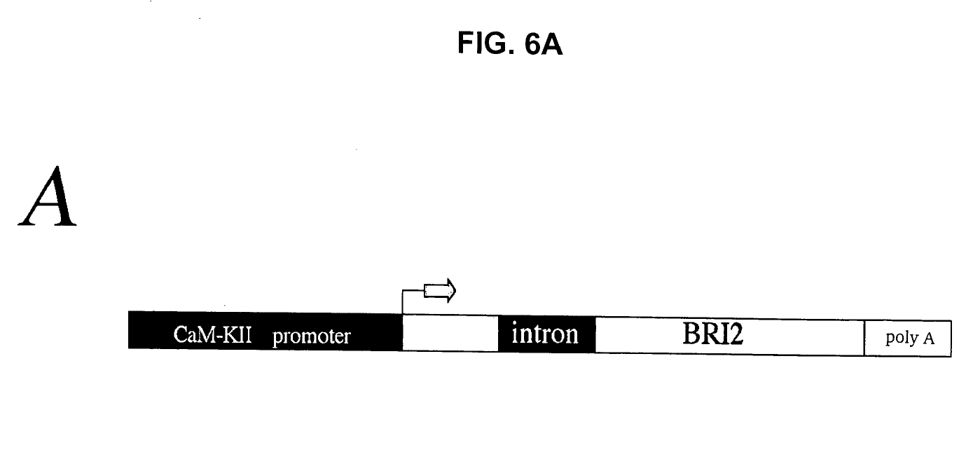

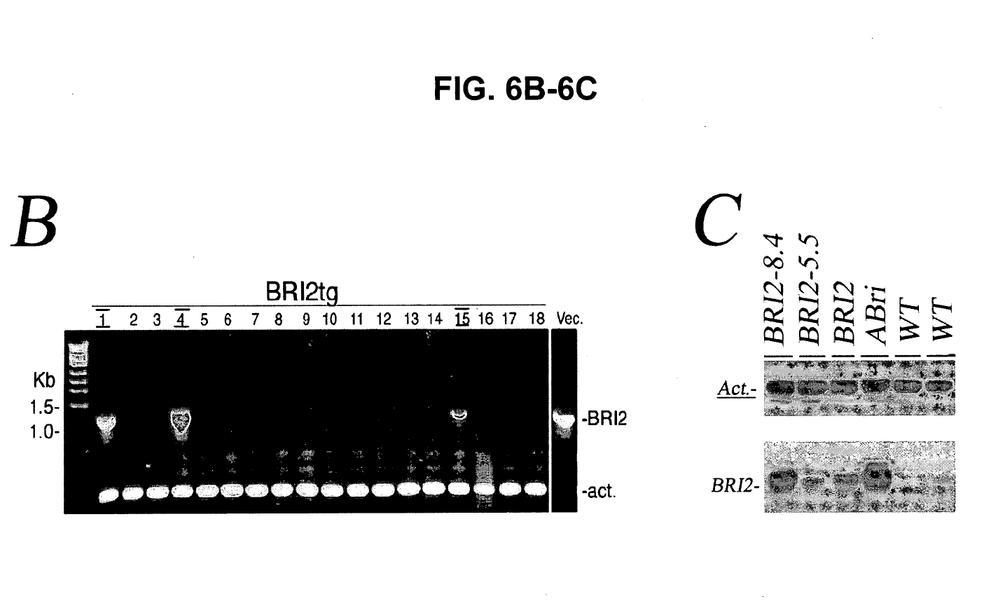

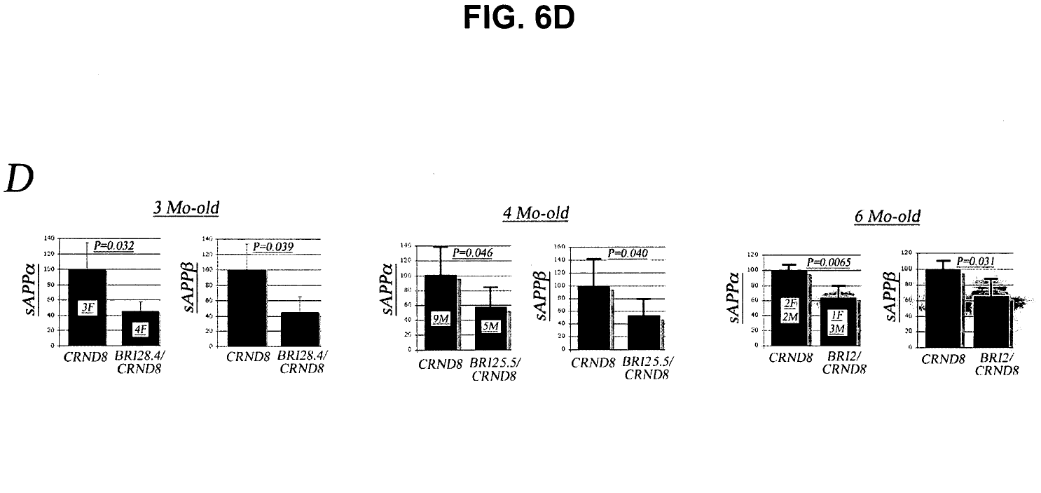

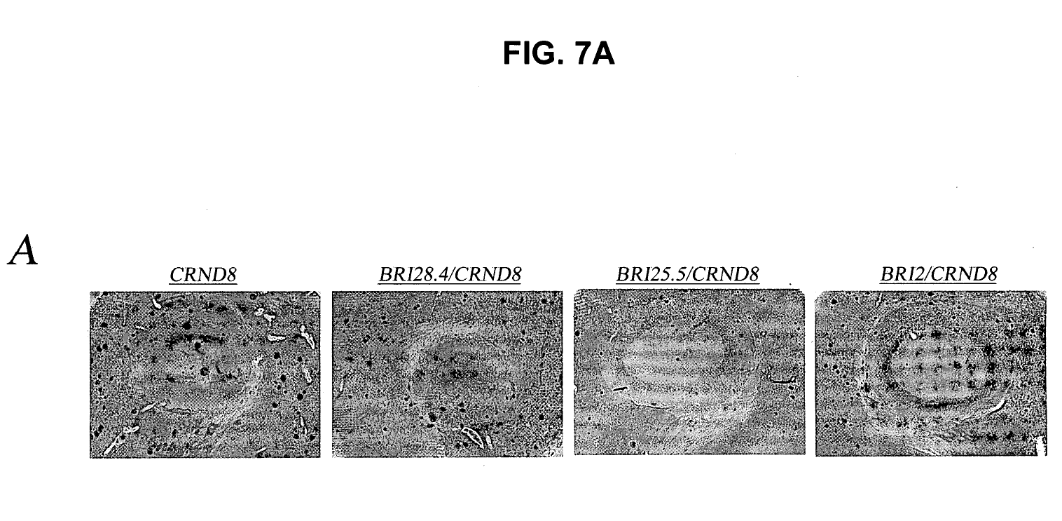

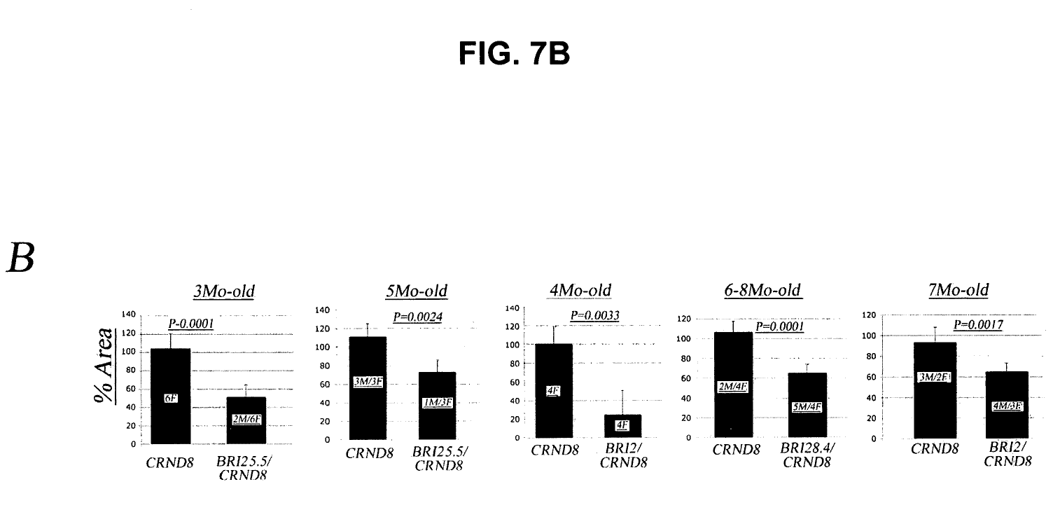

Aβ plaques were also visualized in brain sections from CRND8 or littermate CRND8/BRI2tg animals by immunohistochemically staining with an antibody against Aβ (monoclonal antibody 6E10). Results are shown in FIG. 5. Antibody binding was visualized using a horseradish peroxidase-conjugated secondary antibody and the peroxidase substrate diaminobenzidine. CRND8/BRI2tg animals have reduced size and number of Aβ plaques. Further, two BRI2tg mouse lines BRI2-8.4 and BRI2-5.5, expressing distinct levels of transgenic BRI2 (FIG. 6C), were crossed to CRND8 mice. As an internal control, we also used another transgenic model in which the mouse prion promoter drives BRI2 expression (Pickford et al., 2006). CRND8/BRI2 double transgenic mice and CRND8 single transgenic littermates were analyzed for sAPPα, sAPPβ and amyloid plaque levels. Mice were killed at the indicated ages and brains were isolated. Of importance, transgenic BRI2 expression did not change the levels of transgenic hAPP protein (data not shown). Nevertheless, all three double transgenic mice had significantly reduced sAPPα and sAPPβ levels as compared to littermate CRND8 controls (FIG. 6B). These in vivo data are consistent with the result that BRI2 over expression inhibits both α- and β-secretases, even though we cannot exclude the possibility that the BRI2 transgene also slows the catabolism of sAPPα and sAPPβ in mouse brain. Accordingly, transgenic expression of hBRI2 meaningfully decreased amyloid burden in all three mice lines (FIGS. 7A and B).

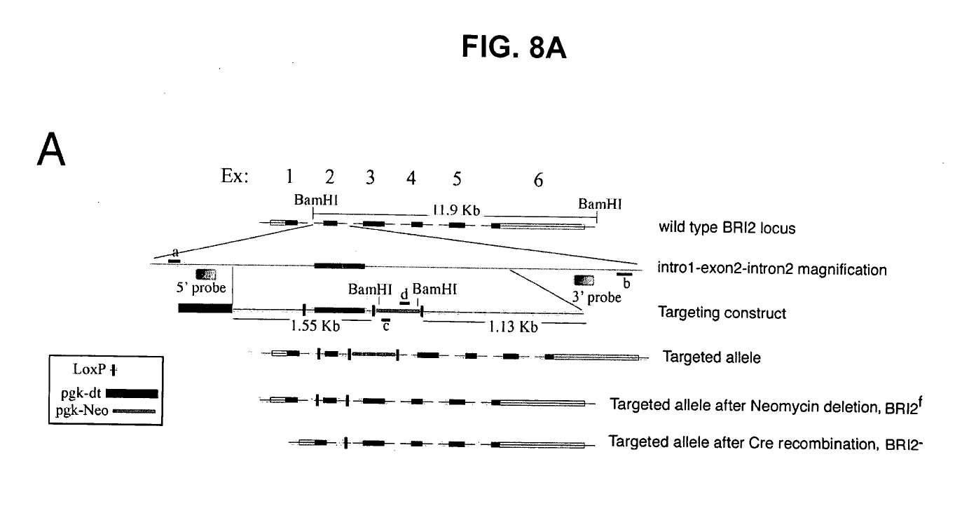

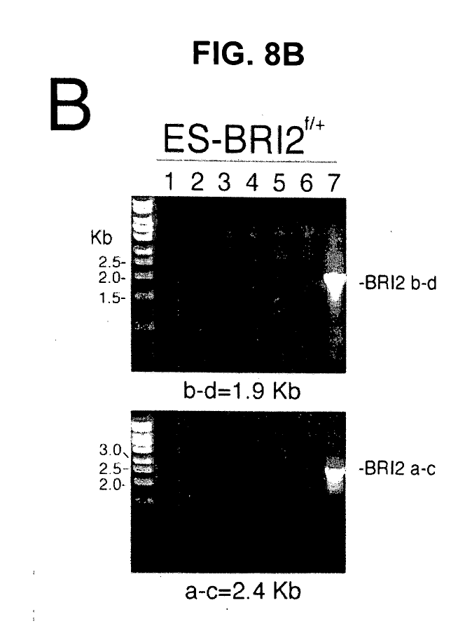

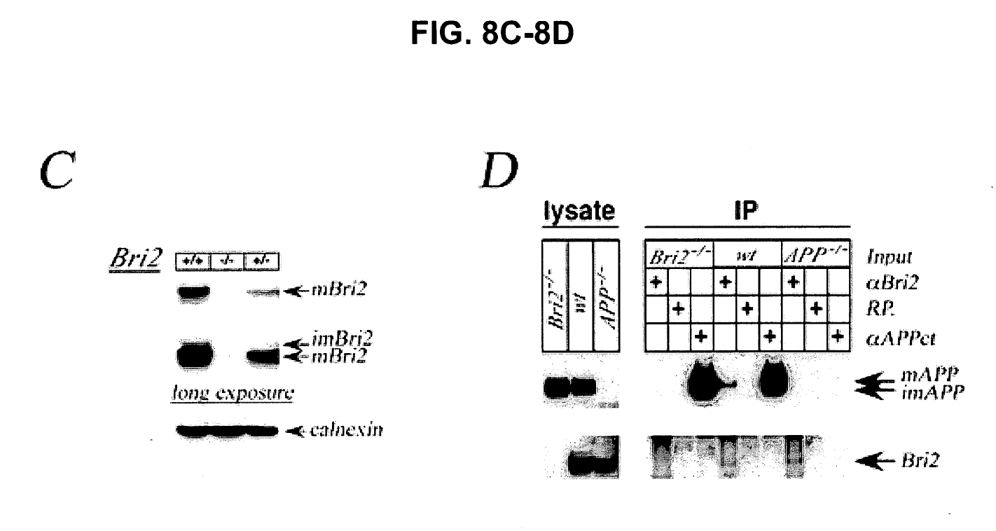

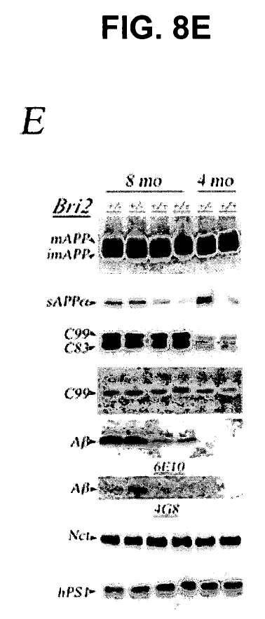

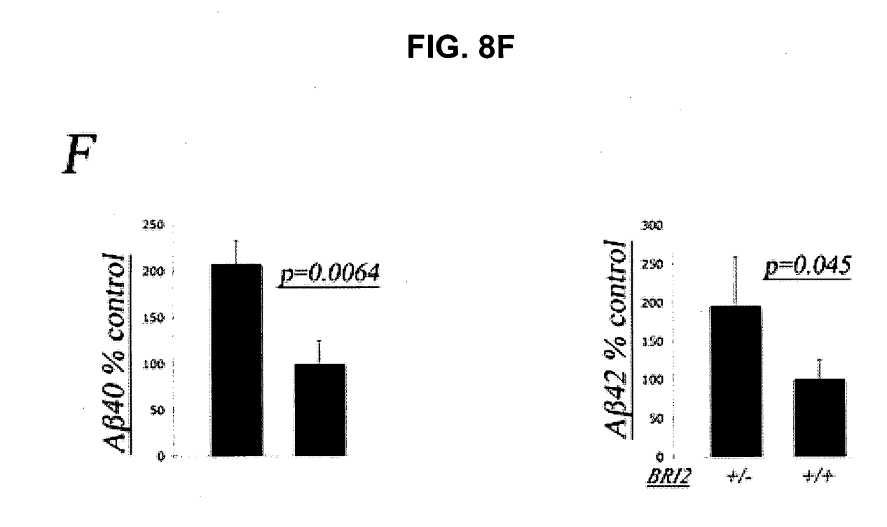

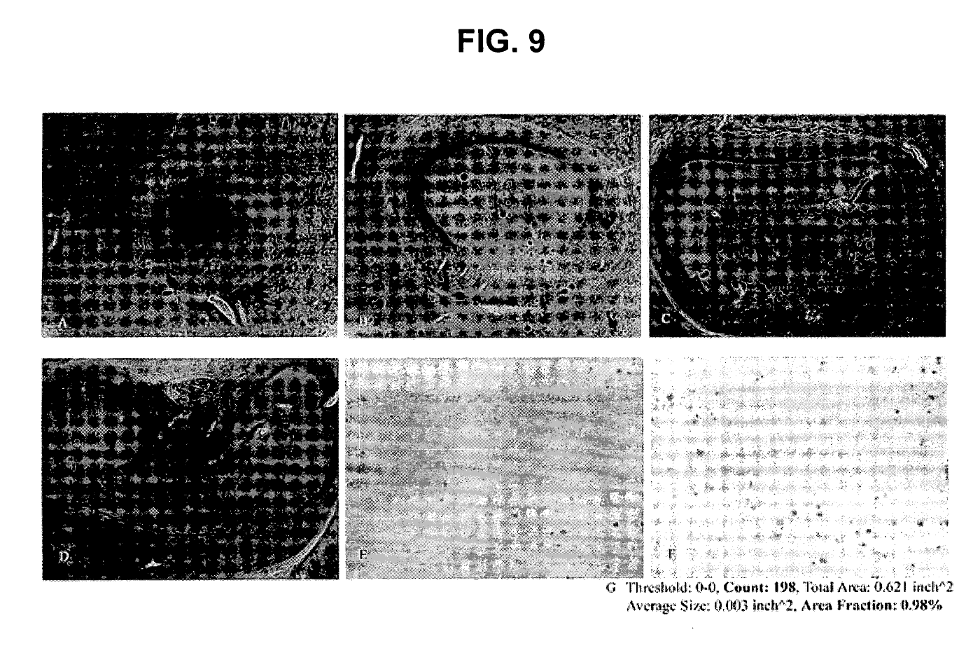

Next we further analyzed Bri2-null mice providing an excellent animal model to validate the role of BRI2 in APP processing (FIG. 8A). To determine how deletion of exon 2 impacts Bri2 protein expression, we performed Western Blot analysis on brain lysates from wild type, Bri2^{-/-} and Bri2^{+/-} APP^{-/-} mice. As expected, Bri2^{-/-} animals did not express Bri2 protein, which is readily detectable in wild type mice. Also, Bri2^{+/-} animals expressed lower levels of Bri2 (FIG. 8C). Analysis of wild type, Bri2^{-/-} and APP^{-/-} mice showed lack of Bri2 expression does not impact on the levels of APP protein and vice versa. Of note, immunoprecipitation of brain lysates with the αBri2 antibody shows that mAPP is bound to APP only in wild type animals, but not in Bri2^{-/-} and APP^{-/-} (FIG. 8D). Bri2-deficient mice were viable and fertile with no obvious changes in overall APP levels as observed by Western blot (FIG. 8C). Bri2^{+/-} mice were crossed to TgAPP695/PSEN1DE9 double transgenic mice (called here APP-PS1) expressing two familial Alzheimer's disease mutant genes (APP-Swedish and PS1DE9) (Savonenko et al., 2003). We analysed two eight month-old and one 4 month-old Bri2^{+/-}/APP-PS1 mice. These mice were compared to age-matched Bri2^{+/+}/APP-PS1. We observed an obvious increase in sAPPα production in brain homogenates of Bri2^{+/-}/APP-PS1 as compared to Bri2^{+/+}/APP-PS1 controls (FIG. 8D). Western blot analysis showed that Aβ is only visible in the eight month-old mice and those Bri2 heterozygous mice present significantly higher levels of Aβ peptides than wild type BRI2 animals (FIG. 8D). To better quantify levels of Aβ40 and Aβ42 we analyzed brain extracts by ELISA. Given the better sensitivity, this assay detected Aβ in the 4 month-old mouse samples as well. These measurements confirmed a statistically significant increase of both Aβ40 and Aβ42 in Bri2^{+/-} as compared to Bri2^{+/+} mice (FIG. 8E). Thus, halving Bri2 expression by gene targeting increases APP processing and Aβ formation.

Besides its biological and pathological relevance, the antiamyloidogenic effect of BRI2 gene therapy in transgenic AD mice (FIG. 7) an the mechanism of inhibition of amyloid formation by BRI2 suggests an alternative approach to AD prevention and/or therapy aimed to inhibiting access of secretases to APP. Molecules mimicking the effect of BRI2 on β-secretase cleavage and/or on γ-secretase docking would reduce Aβ42 formation without affecting secretases' activity on other physiological substrates (Evin et al., 2006). Such drugs would constitute a valid alternative to inhibitor of either γ- or β-secretase, which are currently being developed and tested in clinical trial. β-secretase inhibitors may interfere with peripheral nerve myelination (Hu et al., 2006; Willem et al., 2006), while γ-secretase inhibitors could inhibit a plethora of signaling pathways including but not limited to those mediated by Notch (De Strooper et al., 1999), ErbB4 (Ni et al., 2001), E-Cadherin (Marambaud et al., 2002; Marambaud et al., 2003), p75 (Jung et al., 2003), APLP1 (Scheinfeld et al., 2002), APLP2 (Scheinfeld et al., 2002) and CD44. In addition, recent evidence suggests that FAD-linked Presenilin's mutations may contribute to Alzheimer's disease pathogenesis also with a partial loss-of-function pathogenic mechanism (De Strooper, 2007; Nelson et al., 2007; Saura et al., 2004; Shen and Kelleher, 2007; Tu et al., 2006). These findings raise the valid concern that inhibitors of γ-secretase may be pathogenic rather than therapeutic and accelerate the development of Alzheimer's disease.

### SEQ ID NO:s

### SEQ ID NO:1—Human BRI2 Amino Acid Sequence GenBank Q9Y287

1 mvkvtfnsal aqkeakkdep ksgeealiip pdavavdckd pddvvpvgqr rawcwcmcfg
61 lafmlagvil ggaylykyfa lqpddvyycg ikyikddvil nepsadapaa lyqtieenik
121 ifeeeevefi svpvpefads dpanivhdfn kkltayldln ldkcyvipln tsivmpprnl
181 lellinikag tylpqsylih ehmvitdrie nidhlgffiy rlchdketyk lqrretikgi
241 qkreasncfa irhfenkfav etlics

### SEQ ID NO:2—Human BRI3 Amino Acid Sequence GenBank Q9NQX7.

1 mvkisfqpav agikgdkadk asasapapas ateilltpar eeqppqhrsk rggsvggvcy
61 lsmgmvvllm glvfasvyiy ryfflaqlar dnffrcgvly edslssqvrt qmeleedvki
121 yldenyerin vpvpqfgggd padiihdfqr gltayhdisl dkcyvielnt tivlpprnfw
181 ellmnvkrgt ylpqtyiiqe emvvtehvsd kealgsfiyh lcngkdtyrl rrratrrrin
241 krgakncnai rhfentfvve tlicgvv

### SEQ ID NO:3—Human Furin Preproprotein GenBank NP002560

1 melrpwllwv vaatgtlvll aadaqgqkvf tntwavripg gpavansvar khgflnlgqi
61 fgdyyhfwhr gvtkrslsph rprhsrlqre pqvqwleqqv akrrtkrdvy qeptdpkfpq
121 qwylsgvtqr dlnvkaawaq gytghgivvs ilddgieknh pdlagnydpg asfdvndqdp
181 dpqprytqmn dnrhgtrcag evaavanngv cgvgvaynar iggvrmldge vtdavearsl
241 glnpnhihiy saswgpeddg ktvdgparla eeaffrgvsq grgglgsifv wasgnggreh
301 dscncdgytn siytlsissa tqfgnvpwys eacsstlatt yssgnqnekq ivttdlrqkc
361 teshtgtsas aplaagiial tleanknltw rdmqhlvvqt skpahlnand watngvgrkv
421 shsygyglld agamvalaqn wttvapqrkc iidiltepkd igkrlevrkt vtaclgepnh
481 itrlehaqar ltlsynrrgd laihlvspmg trstllaarp hdysadgfnd wafmtthswd
541 edpsgewvle ientseanny gtltkftlvl ygtapeglpv ppessgcktl tssqacvvce
601 egfslhqksc vqhcppgfap qvldthyste ndvetirasv capchascat cqgpaltdcl
661 scpshasldp veqtcsrqsq ssresppqqq pprlppevea gqrlragllp shlpevvagl
721 scafivlvfv tvflvlqlrs gfsfrgvkvy tmdrglisyk glppeawqee cpsdseedeg
781 rgertafikd qsal

### REFERENCES

Akiyama H, Kondo H, Arai T, Ikeda K, Kato M, Iseki E, Schwab C and McGeer P L, Expression of BRI, the normal precursor of the amyloid protein of familial British dementia, in human brain. Acta Neuropathol (Berl) 107(1): 53-8, 2004.
Breteler M M, Claus J J, van Duijn C M, Launer L J and Hofman A, Epidemiology of Alzheimer's disease. Epidemiol Rev 14: 59-82, 1992.
Cao X and Sudhof T C, A transcriptionally [correction of transcriptively] active complex of APP with Fe65 and histone acetyltransferase Tip60. Science 293(5527): 115-20, 2001.
Cupers P, Orlans I, Craessaerts K, Annaert W and De Strooper B, The amyloid precursor protein (APP)-cytoplasmic fragment generated by gamma-secretase is rapidly degraded but distributes partially in a nuclear fraction of neurones in culture. J Neurochem 78(5): 1168-78, 2001.
De Strooper B, Saftig P, Craessaerts K, Vanderstichele H, Guhde G, Annaert W, Von Figura K and Van Leuven F, Deficiency of presenilin-1 inhibits the normal cleavage of amyloid precursor protein. Nature 391(6665): 387-90, 1998.
De Strooper B, Annaert W, Cupers P, Saftig P, Craessaerts K, Mumm J S, Schroeter E H, Schrijvers V, Wolfe M S, Ray W J, Goate A and Kopan R, A presenilin-1-dependent gamma-secretase-like protease mediates release of Notch intracellular domain. Nature 398(6727): 518-22, 1999.
De Strooper, B. (2007). Loss-of-function presenilin mutations in Alzheimer disease. Talking Point on the role of presenilin mutations in Alzheimer disease. EMBO Rep 8, 141-146.
Evin, G., Sernee, M. F., and Masters, C. L. (2006). Inhibition of gamma-secretase as a therapeutic intervention for Alzheimer's disease: prospects, limitations and strategies. CNS Drugs 20, 351-372.
Fotinopoulou A, Tsachaki M, Vlavaki M, Poulopoulos A, Rostagno A, Frangione B, Ghiso J and Efthimiopoulos S, BRI2 interacts with amyloid precursor protein (APP) and regulates amyloid beta (Abeta) production. J Biol Chem 280(35): 30768-72, 2005.
Francis R, McGrath G, Zhang J, Ruddy D A, Sym M, Apfeld J, Nicoll M, Maxwell M, Hai B, Ellis M C, Parks A L, Xu W, Li J, Gurney M, Myers R L, Himes C S, Hiebsch R, Ruble C, Nye J S and Curtis D, aph-1 and pen-2 are required for Notch pathway signaling, gamma-secretase cleavage of betaAPP, and presenilin protein accumulation. Dev Cell 3(1): 85-97, 2002.
Glenner G G, Wong C W, Quaranta V and Eanes E D, The amyloid deposits in Alzheimer's disease: their nature and pathogenesis. Appl Pathol 2(6): 357-69, 1984.
Glenner G G and Wong C W, Alzheimer's disease and Down's syndrome: sharing of a unique cerebrovascular amyloid fibril protein. Biochem Biophys Res Commun 122(3): 1131-5, 1984.
Goate A, Chartier-Harlin M C, Mullan M, Brown J, Crawford F, Fidani L, Giuffra L, Haynes A, Irving N, James L and et al., Segregation of a missense mutation in the amyloid precursor protein gene with familial Alzheimer's disease. Nature 349(6311): 704-6, 1991.
Goutte C, Tsunozaki M, Hale V A and Priess J R, APH-1 is a multipass membrane protein essential for the Notch signaling pathway in Caenorhabditis elegans embryos. Proc Natl Acad Sci U S A 99(2): 775-9, 2002.
Hu, X., Hicks, C. W., He, W., Wong, P., Macklin, W. B., Trapp, B. D., and Yan, R. (2006). Bace1 modulates myelination in the central and peripheral nervous system. Nat Neurosci 9, 1520-1525.
Janus C, Pearson J, McLaurin J, Mathews P M, Jiang Y, Schmidt S D, Chishti M A, Horne P, Heslin D, French J, Mount H T, Nixon R A, Mercken M, Bergeron C, Fraser P E, St George-Hyslop P and Westaway D, A beta peptide immunization reduces behavioural impairment and plaques in a model of Alzheimer's disease. Nature 408(6815): 979-82, 2000.
Kim S H, Wang R, Gordon D J, Bass J, Steiner D F, Lynn D G, Thinakaran G, Meredith S C and Sisodia S S, Furin mediates enhanced production of fibrillogenic ABri peptides in familial British dementia. Nat Neurosci 2(11): 984-8, 1999.
Lammich S, Okochi M, Takeda M, Kaether C, Capell A, Zimmer A K, Edbauer D, Walter J, Steiner H and Haass C, Presenilin-dependent intramembrane proteolysis of CD44 leads to the liberation of its intracellular domain and the secretion of an Abeta-like peptide. J Biol Chem 277(47): 44754-9, 2002.
Levy-Lahad E, Wijsman E M, Nemens E, Anderson L, Goddard K A, Weber J L, Bird T D and Schellenberg G D, A familial Alzheimer's disease locus on chromosome 1. Science 269(5226): 970-3, 1995a.
Levy-Lahad E, Wasco W, Poorkaj P, Romano D M, Oshima J, Pettingell W H, Yu C E, Jondro P D, Schmidt S D, Wang K and et al., Candidate gene for the chromosome 1 familial Alzheimer's disease locus. Science 269(5226): 973-7, 1995b.
Marambaud P, Shioi J, Serban G, Georgakopoulos A, Sarner S, Nagy V, Baki L, Wen P, Efthimiopoulos S, Shao Z, Wisniewski T and Robakis N K, A presenilin-1/gamma-secretase cleavage releases the E-cadherin intracellular domain and regulates disassembly of adherens junctions. Embo J 21(8): 1948-56, 2002.
Marambaud P, Wen P H, Dutt A, Shioi J, Takashima A, Siman R and Robakis N K, A CBP binding transcriptional repressor produced by the PS1/epsilon-cleavage of N-cadherin is inhibited by PS1 FAD mutations. Cell 114(5): 635-45, 2003.
Matsuda S, Giliberto L, Matsuda Y, Davies P, McGowan E, Pickford F, Ghiso J, Frangione B and D'Adamio L, The familial dementia BRI2 gene binds the Alzheimer gene amyloid-beta precursor protein and inhibits amyloid-beta production. J Biol Chem 280(32): 28912-6, 2005.
Mayford M, Bach M E and Kandel E, CaMKII function in the nervous system explored from a genetic perspective. Cold Spring Harb Symp Quant Biol 61: 219-24, 1996.
Nelson, O., Tu, H., Lei, T., Bentahir, M., de Strooper, B., and Bezprozvanny, I. (2007). Familial Alzheimer disease-linked mutations specifically disrupt Ca2+ leak function of presenilin 1. J Clin Invest 117, 1230-1239.
Ni C Y, Murphy M P, Golde T E and Carpenter G, gamma-Secretase cleavage and nuclear localization of ErbB-4 receptor tyrosine kinase. Science 294(5549): 2179-81, 2001.
Passer B, Pellegrini L, Russo C, Siegel R M, Lenardo M J, Schettini G, Bachmann M, Tabaton M and D'Adamio L, Generation of an apoptotic intracellular peptide by gamma-secretase cleavage of Alzheimer's amyloid beta protein precursor. J Alzheimers Dis 2(3-4): 289-301, 2000.
Phinney A L, Drisaldi B, Schmidt S D, Lugowski S, Coronado V, Liang Y, Horne P, Yang J, Sekoulidis J, Coomaraswamy J, Chishti M A, Cox D W, Mathews P M, Nixon R A, Carlson G A, St George-Hyslop P and Westaway D, In vivo reduction of amyloid-β by a mutant copper transporter. Proc Natl Acad Sci U S A. 100(24): 14193-14198, 2003.
Pickford F, Coomaraswamy J, Jucker M, McGowan E, Modeling Familial British Dementia in Transgenic Mice. Brain Pathol. 16: 80-85, 2006.
Prasher V P, Farrer M J, Kessling A M, Fisher E M, West R J, Barber P C and Butler A C, Molecular mapping of Alzheimer-type dementia in Down's syndrome. Ann Neurol 43(3): 380-3, 1998.
Revesz T, Holton J L, Lashley T, Plant G, Rostagno A, Ghiso J and Frangione B, Sporadic and familial cerebral amyloid angiopathies. Brain Pathol 12(3): 343-57, 2002.
Rogaev E I, Sherrington R, Rogaeva E A, Levesque G, Ikeda M, Liang Y, Chi H, Lin C, Holman K, Tsuda T and et al., Familial Alzheimer's disease in kindreds with missense mutations in a gene on chromosome 1 related to the Alzheimer's disease type 3 gene. Nature 376(6543): 775-8, 1995.
Saura, C. A., Choi, S. Y., Beglopoulos, V., Malkani, S., Zhang, D., Shankaranarayana Rao, B. S., Chattarji, S., Kelleher, R. J., 3rd, Kandel, E. R., Duff, K., et al. (2004). Loss of presenilin function causes impairments of memory and synaptic plasticity followed by age-dependent neurodegeneration. Neuron 42, 23-36.
Savonenko, A. V., Xu, G. M., Price, D. L., Borchelt, D. R., and Markowska, A. L. (2003). Normal cognitive behavior in two distinct congenic lines of transgenic mice hyperexpressing mutant APP SWE. Neurobiol Dis 12, 194-211.
St George-Hyslop P H, Haines J L, Farrer L A, Polinsky R, Van Broeckhoven C, Goate A, McLachlan D R, Orr H, Bruni A C, Sorbi S and et al., Genetic linkage studies suggest that Alzheimer's disease is not a single homogeneous disorder. FAD Collaborative Study Group. Nature 347(6289): 194-7, 1990.
Scheinfeld M H, Ghersi E, Laky K, Fowlkes B J and D'Adamio L, Processing of beta-amyloid precursor-like protein-1 and -2 by gamma-secretase regulates transcription. J Biol Chem 277(46): 44195-201, 2002.
Schellenberg G D, Payami H, Wijsman E M, Orr H T, Goddard K A, Anderson L, Nemens E, White J A, Alonso M E; Ball M J and et al., Chromosome 14 and late-onset familial Alzheimer disease (FAD). Am J Hum Genet 53(3): 619-28, 1993.
Selkoe D J and Podlisny M B, Deciphering the genetic basis of Alzheimer's disease. Annu Rev Genomics Hum Genet 3: 67-99, 2002.
Selkoe D and Kopan R, Notch and Presenilin: regulated intramembrane proteolysis links development and degeneration. Annu Rev Neurosci 26: 565-97, 2003.
Shen, J., and Kelleher, R. J., 3rd (2007). The presenilin hypothesis of Alzheimer's disease: evidence for a loss-of-function pathogenic mechanism. Proc Natl Acad Sci USA 104, 403-409.
Sherrington R, Rogaev E I, Liang Y, Rogaeva E A, Levesque G, Ikeda M, Chi H, Lin C, Li G, Holman K and et al., Cloning of a gene bearing missense mutations in early-onset familial Alzheimer's disease. Nature 375(6534): 754-60, 1995.
Sisodia S S and St George-Hyslop P H, gamma-Secretase, Notch, Abeta and Alzheimer's disease: where do the presenilins fit in? Nat Rev Neurosci 3(4): 281-90, 2002.
Stagljar I, Korostensky C, Johnsson N and te Heesen S, A genetic system based on split-ubiquitin for the analysis of interactions between membrane proteins in vivo. Proc Natl Acad Sci USA 95(9): 5187-92, 1998.
Tomidokoro Y, Lashley T, Rostagno A, Neubert T, Bojsen-Moller M, Braendgaard H, Plant G, Holton J, Frangione B, Revesz T and Ghiso J, Familial Danish dementia: Co-existence of Danish and Alzheimer amyloid subunits (ADan and Abeta) in the absence of compact plaques. J Biol Chem, 2005.
Tu, H., Nelson, O., Bezprozvanny, A., Wang, Z., Lee, S. F., Hao, Y. H., Serneels, L., De Strooper, B., Yu, G., and Bezprozvanny, I. (2006). Presenilins form ER Ca2+ leak channels, a function disrupted by familial Alzheimer's disease-linked mutations. Cell 126, 981-993.
van Duijn C M, Clayton D, Chandra V, Fratiglioni L, Graves A B, Heyman A, Jorm A F, Kokmen E, Kondo K, Mortimer J A and et al., Familial aggregation of Alzheimer's disease and related disorders: a collaborative re-analysis of case-control studies. EURODEM Risk Factors Research Group. Int J Epidemiol 20 Suppl 2: S13-20, 1991.
Vassar R, Bennett B D, Babu-Khan S, Kahn S, Mendiaz E A, Denis P, Teplow D B, Ross S, Amarante P, Loeloff R, Luo Y, Fisher S, Fuller J, Edenson S, Lile J, Jarosinski M A, Biere A L, Curran E, Burgess T, Louis J C, Collins F, Treanor J, Rogers G and Citron M, Beta-secretase cleavage of Alzheimer's amyloid precursor protein by the transmembrane aspartic protease BACE. Science 286(5440): 735-41, 1999.
Vidal R, Frangione B, Rostagno A, Mead S, Revesz T, Plant G and Ghiso J, A stop-codon mutation in the BRI gene associated with familial British dementia. Nature 399(6738): 776-81, 1999.
Vidal R, Revesz T, Rostagno A, Kim E, Holton J L, Bek T, Bojsen-Moller M, Braendgaard H, Plant G, Ghiso J and Frangione B, A decamer duplication in the 3′ region of the BRI gene originates an amyloid peptide that is associated with dementia in a Danish kindred. Proc Natl Acad Sci USA 97(9): 4920-5, 2000.
Willem M, Garratt A M, Novak B, Citron M, Kaufmann S, Rittger A, DeStrooper B, Saftig P, Birchmeier C, Haass C, Control of Peripheral Nerve Myelination by the β-Secretase BACE1. Science Published Online Sep. 21, 2006, DOI: 10.1126/science. 1132341.
Yu G, Nishimura M, Arawaka S, Levitan D, Zhang L, Tandon A, Song Y Q, Rogaeva E, Chen F, Kawarai T, Supala A, Levesque L, Yu H, Yang D S, Holmes E, Milman P, Liang Y, Zhang D M, Xu D H, Sato C, Rogaev E, Smith M, Janus C, Zhang Y, Aebersold R, Farrer L S, Sorbi S, Bruni A, Fraser P and St George-Hyslop P, Nicastrin modulates presenilin-mediated notch/glp-1 signal transduction and betaAPP processing. Nature 407(6800): 48-54, 2000.
Zazzeroni F, Papa S, Algeciras-Schimnich A, Alvarez K, Melis T, Bubici C, Majewski N, Hay N, De Smaele E, Peter M E and Franzoso G, Gadd45 beta mediates the protective effects of CD40 costimulation against Fas-induced apoptosis. Blood 102(9): 3270-9, 2003.
PCT Patent Application No. PCT/US06/23135.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A non-human mammal comprising a nucleic acid sequence capable of causing an alteration of expression of Bri2 or Bri3 in the mammal, wherein the mammal is a model for Alzheimer's disease.

2. The mammal of claim 1, wherein the sequence comprises a segment encoding at least a portion of the Bri2 or Bri3 protein at least 80% homologous to SEQ ID NO:1 or SEQ ID NO:2.

3-4. (canceled)

5. The mammal of claim 2, wherein the Bri2 or Bri3 protein is a wild-type Bri2 or Bri3 protein.

6. The mammal of claim 2, wherein the Bri2 or Bri3 protein is a human protein.

7. The mammal of claim 2, wherein the segment comprises a Bri2 gene with a mutation in the stop codon allowing translational read-through as with a human Bri2 gene associated with Familial British Dementia (FBD).

8. The mammal of claim 7, wherein the segment encodes a human Bri2 protein associated with Familial British Dementia (FBD).

9. The mammal of claim 2, wherein the segment comprises a Bri2 gene with a decamer duplication in the 3′ region as with the human gene associated with Familial Danish Dementia (FDD).

10. The mammal of claim 9, wherein the segment encodes a human Bri2 protein associated with FDD.

11. The mammal of claim 1, wherein the sequence is an insert into, or a replacement of, at least a portion of a native Bri2 or Bri3 gene.

12. The mammal of claim 11, wherein the insert or replacement deletes the native BRI2 exon 2.

13-15. (canceled)

16. The mammal of claim 1, wherein the alteration of expression of Bri2 or Bri3 in the mammal is conditional.

17. (canceled)

18. The mammal of claim 11, wherein the sequence comprises a non-Bri sequence causing a knockout of the Bri gene.

19-32. (canceled)

33. The mammal of claim 1, wherein the mammal is a mouse and the sequence comprises a LoxP site such that exon 2 of the Bri2 gene is deleted upon induction of Cre-mediated recombination.

34. The mammal of claim 1, wherein the mammal is a mouse and the sequence comprises a Bri2 exon 6 homologously inserted into the mouse Bri2 gene, wherein the Bri2 exon 6 comprises a mutation in the stop codon allowing translational read-through as with a human Bri2 gene associated with Familial British Dementia (FBD).

35. The mammal of claim 1, wherein the mammal is a mouse and the sequence comprises a Bri2 exon 6 homologously inserted into the mouse Bri2 gene, wherein the Bri2 exon 6 comprises a decamer duplication as with the human gene associated with Familial Danish Dementia (FDD).

36. A non-human mammal comprising a Bri2 or Bri3 gene under the control of the native Bri2 or Bri3 promoter, wherein the Bri2 or Bri3 gene does not naturally occur in the mammal.

37-43. (canceled)

44. A non-human mammal genetically engineered to lack expression of a Bri2 or Bri3 gene.

45. (canceled)

46. The mammal of claim 44, wherein the mammal is a model for Alzheimer's disease after alteration.

47-61. (canceled)

62. The mammal of claim 1, wherein the mammal is heterozygous for the haplotype.

63. The mammal of claim 1, wherein the mammal is homozygous for the haplotype.

64-65. (canceled)

66. The mammal of claim 1, showing a reduced cognitive ability over the mammal without the transgenic nucleic acid sequence.

67. An embryonic stem cell of the mammal from claim 1.

68. A somatic cell from the mammal of claim 1.

69-72. (canceled)

73. A method of screening a compound for treatment of a disease characterized by cerebral amyloidosis, dementia, and/or cognitive impairment, the method comprising administering the compound to any one of the mammals of claim 1 that has cerebral amyloidosis, dementia, and/or cognitive impairment, then determining whether the compound affects the cerebral amyloidosis, dementia, and/or cognitive impairment.

74-81. (canceled)

82. The method of claim 73, wherein determining whether the compound affects the cerebral amyloidosis, dementia, and/or cognitive impairment is performed by determining whether the compound increases a cognitive ability of the mammal.

83-86. (canceled)

87. A method of screening a compound for treatment of a disease characterized by cerebral amyloidosis, dementia, and/or cognitive impairment, the method comprising administering the compound to a neuron of claim 68 from a mammal that has cerebral amyloidosis, dementia, and/or cognitive impairment, then determining whether the compound affects ABri, Aβ or ADAN production.

88-106. (canceled)

107. A method of making a transgenic non-human mammal, the method comprising
(a) transfecting embryonic stem cells of the mammal with a transgenic nucleic acid sequence capable of causing an alteration of expression of Bri2 or Bri3 in the mammal;
(b) injecting the transfected embryonic stem cells into blastocysts of the mammal and implanting the blastocysts into the uterus of a foster mother of the mammal;
(c) raising pups from the foster mother; and
(d) identifying a transgenic pup, which is the transgenic non-human mammal, wherein the transgenic non-human mammal does not express a Bri2 or Bri3.

108-125. (canceled)
